(19) United States
(12) Patent Application Publication
(10) Pub. No.: US 2023/0233138 A1
(43) Pub. Date: Jul. 27, 2023

(54) TECHNIQUE FOR IDENTIFYING DEMENTIA BASED ON PLURALITY OF RESULT DATA

(71) Applicant: HAII CORP., Seoul (KR)

(72) Inventors: Ho Yung KIM, Seoul (KR); Bo Hee KIM, Seoul (KR); Dong Han KIM, Seoul (KR); Hye Bin HWANG, Incheon (KR); Chan Yeong PARK, Seoul (KR); Ji An CHOI, Seoul (KR)

(21) Appl. No.: 18/082,404

(22) Filed: Dec. 15, 2022

(30) Foreign Application Priority Data
Jan. 21, 2022 (KR) 10-2022-0008987

(51) Int. Cl. A61B 5/00; G16H 50/20; G16H 10/60

(52) U.S. Cl. CPC A61B 5/4088; G16H 50/20; G16H 10/60

(57) ABSTRACT

Disclosed is a method of identifying dementia by at least one processor of a device according to some embodiments of the present disclosure. The method may include obtaining a plurality of result data of a user obtained by performing a plurality of tests through a user terminal, calculating a score value by inputting the plurality of result data to a dementia identification model, and determining whether the user has dementia based on whether the score value is greater than a first threshold value.

### BACKGROUND

### 1. Technical Field

The present disclosure relates to a technique for identifying dementia based on a plurality of result data, and particularly, to an apparatus and method for identifying dementia by using a plurality of result data generated based on a plurality of tests as a digital biomarker.

### 2. Related Art

Alzheimer's disease (AD), which is a brain disease caused by aging, causes progressive memory impairment, cognitive deficits, changes in individual personality, etc. In addition, dementia refers to a state of persistent and overall cognitive function decline that occurs when a person who has led a normal life suffers from damage to brain function due to various causes. In this case, cognitive function refers to various intellectual abilities, such as memory, language ability, temporal and spatial understanding ability, judgment ability, and abstract thinking ability. Each cognitive function is closely related to a specific part of the brain. The most common form of dementia is Alzheimer's disease.

Various methods have been proposed for diagnosing Alzheimer's disease, dementia, or mild cognitive impairment. For example, a method of diagnosing Alzheimer's disease or mild cognitive impairment using the expression level of miR-206 in the olfactory tissue, and a method for diagnosing dementia using a biomarker that characteristically increases in blood are known.

However, since special equipment or tests necessary for biopsy are required so as to use miR-206 in the olfactory tissue, and blood from a patient should be collected by an invasive method so as to use biomarkers in blood, there is a disadvantage that the patient's rejection feeling is relatively large.

Therefore, there is an urgent need for development of a dementia diagnosis method where patients hardly feel rejection without a separate special equipment or examination.

### SUMMARY

The present disclosure has been made in view of the above problems, and it is one object of the present disclosure to provide an accurate dementia diagnosis method where patients hardly feel rejection.

It will be understood that technical problems of the present disclosure are not limited to the aforementioned problem and other technical problems not referred to herein will be clearly understood by those skilled in the art from the description below.

In an embodiment, a method of identifying, by at least one processor of a device, dementia may include obtaining a plurality of result data of a user obtained by performing a plurality of tests through a user terminal, calculating a score value by inputting the plurality of result data to a dementia identification model, and determining whether the user has dementia based on whether the score value is greater than a first threshold value.

According to some embodiments of the present disclosure, the plurality of tests may include at least one of a Stroop test, a calculation ability test, a memory test, a gaze test, and a mixed test.

According to some embodiments of the present disclosure, the plurality of tests may be performed in a way to display at least one element along with the output of sound data and message data that explain a method of performing each of the plurality of tests.

According to some embodiments of the present disclosure, the determining of whether the user has dementia based on whether the score value is greater than the first threshold value may include determining that the user has dementia when the score value is greater than the first threshold value, determining that the user has mild cognitive impairment (MCI) when the score value is greater than a second threshold value and is smaller than or equal to the first threshold value, or determining that the user is normal when the score value is smaller than or equal to the second threshold value.

According to some embodiments of the present disclosure, the determining that the user has MCI may include causing an application for improving cognitive power of the user to be executed in or downloaded to the user terminal.

According to some embodiments of the present disclosure, the determining of whether the user has dementia may further include causing dementia identification result information to be output through a preset application of the user terminal of the user.

According to some embodiments of the present disclosure, the result information may include current state information and state change information of the user that are generated based on history data of the user that was obtained by performing the plurality of tests and current data of the user that is now obtained by performing the plurality of tests.

According to some embodiments of the present disclosure, the method may further include causing hospital information generated based on information on an address of the user to be output when the score value is greater than the first threshold value.

According to some embodiments of the present disclosure, the method may further include obtaining information on an age and sex of the user from the user terminal before obtaining the plurality of result data. The calculating of the score value by inputting the plurality of result data to the dementia identification model may include calculating the score value by inputting the information on the age and sex to the dementia identification model along with the plurality of result data.

According to some embodiments of the present disclosure, the dementia identification model may include a plurality of sub-models for receiving the plurality of result data, respectively. The score value may be an average value of a plurality of sub-score values output by the plurality of sub-models, respectively.

According to some embodiments of the present disclosure, the dementia identification model may include a plurality of sub-models for receiving the plurality of result data, respectively. The calculating of the score value by inputting the plurality of result data to the dementia identification model may include adding a weight of each of the plurality of sub-models to each of a plurality of sub-score values output by the plurality of sub-models, and determining, as the score value, an average value of the plurality of sub-score values to which the weights have been added.

According to some embodiments of the present disclosure, the score value or dementia identification result information of the user may be transmitted to an external server in order to calculate dementia-related insurance premium of the user.

According to some embodiments of the present disclosure, a computer program in which a non-transitory computer-readable storage medium has been stored performs identifying dementia when the computer program is executed in at least one processor of a device. The identifying of the dementia may include obtaining a plurality of result data of a user obtained by performing a plurality of tests through a user terminal, calculating a score value by inputting the plurality of result data to a dementia identification model, and determining whether the user has dementia based on whether the score value is greater than a first threshold value.

According to some embodiments of the present disclosure, a device for identifying dementia includes storage in which at least one program instruction has been stored and at least one processor configured to perform the at least one program instruction. The at least one processor may obtain a plurality of result data of a user obtained by performing a plurality of tests through a user terminal, may calculate a score value by inputting the plurality of result data to a dementia identification model, and may determine whether the user has dementia based on whether the score value is greater than a first threshold value.

The effect of a technique of identifying dementia according to the present disclosure is as follows.

According to some embodiments of the present disclosure, an accurate dementia diagnosis method where patients hardly feel rejection is provided.

It will be understood that effects obtained by the present disclosure are not limited to the aforementioned effect and other effects not referred to herein will be clearly understood by those skilled in the art from the description below.

### BRIEF DESCRIPTION OF THE DRAWINGS

Various embodiments of the present disclosure are described with reference to the accompanying drawings. In this case, like reference numbers are used to refer to like elements. In the following embodiments, numerous specific details are set forth so as to provide a thorough understanding of one or more embodiments for purposes of explanation. It will be apparent, however, that such embodiment(s) may be practiced without these specific details.

FIG. 1 is a schematic diagram for explaining a system for identifying dementia according to some embodiments of the present disclosure.

FIG. 2 is a flowchart for describing an embodiment of a method of identifying whether a user has dementia according to some embodiments of the present disclosure.

FIG. 3 is a diagram for describing an embodiment of a method of obtaining the geometrical features of an eye of a user according to some embodiments of the present disclosure.

FIG. 4 is a diagram for describing an embodiment of a method of performing a Stroop test according to some embodiments of the present disclosure.

FIG. 5 is a diagram for describing an embodiment of a method of performing a calculation ability test according to some embodiments of the present disclosure.

FIG. 6 is a diagram for describing an embodiment of a method of performing a memory test according to some embodiments of the present disclosure.

FIGS. 7 to 9 are diagrams for describing an embodiment of a method of performing a gaze test according to some embodiments of the present disclosure.

FIG. 10 is a diagram for describing another embodiment of a method of performing a memory test according to some embodiments of the present disclosure.

FIGS. 11 to 13 are diagrams for describing another embodiment of a method of performing a mixed test according to some embodiments of the present disclosure.

FIG. 14 is a diagram for describing an embodiment of a method of displaying dementia identification result information through a preset application according to some embodiments of the present disclosure.

### DETAILED DESCRIPTION

Hereinafter, various embodiments of an apparatus according to the present disclosure and a method of controlling the same will be described in detail with reference to the accompanying drawings. Regardless of the reference numerals, the same or similar components are assigned the same reference numerals, and overlapping descriptions thereof will be omitted.

Objectives and effects of the present disclosure, and technical configurations for achieving the objectives and the effects will become apparent with reference to embodiments described below in detail in conjunction with the accompanying drawings. In describing one or more embodiments of the present disclosure, a detailed description of known functions and configurations incorporated herein will be omitted when it may make the subject matter of the present disclosure unclear.

The terms used in the specification are defined in consideration of functions used in the present disclosure, and can be changed according to the intent or conventionally used methods of clients, operators, and users. The features of the present disclosure will be more clearly understood from the accompanying drawings and should not be limited by the accompanying drawings, and it is to be appreciated that all changes, equivalents, and substitutes that do not depart from the spirit and technical scope of the present disclosure are encompassed in the present disclosure.

The suffixes “module” and “unit” of elements herein are used for convenience of description and thus can be used interchangeably and do not have any distinguishable meanings or functions.

Terms including an ordinal number, such as first, second, etc., may be used to describe various elements, but the elements are not limited by the terms. The above terms are used only for the purpose of distinguishing one component from another component. Therefore, a first component mentioned below may be a second component within the spirit of the present description.

A singular expression includes a plural expression unless the context clearly dictates otherwise. That is, a singular expression in the present disclosure and in the claims should generally be construed to mean “one or more” unless specified otherwise or if it is not clear from the context to refer to a singular form.

The terms such as “include” or “comprise” may be construed to denote a certain characteristic, number, step, operation, constituent element, or a combination thereof, but may not be construed to exclude the existence of or a possibility of addition of one or more other characteristics, numbers, steps, operations, constituent elements, or combinations thereof.

The term “or” in the present disclosure should be understood as “or” in an implicit sense and not “or” in an exclusive sense. That is, unless otherwise specified or clear from context, “X employs A or B” is intended to mean one of natural implicit substitutions. That is, when X employs A; when X employs B; or when X employs both A and B, “X employs A or B” can be applied to any one of these cases. Furthermore, the term “and/or” as used in the present disclosure should be understood to refer to and encompass all possible combinations of one or more of listed related items.

As used in the present disclosure, the terms “information” and “data” may be used interchangeably.

Unless otherwise defined, all terms (including technical and scientific terms) used in the present disclosure may be used with meanings that can be commonly understood by those of ordinary skill in the technical field of the present disclosure. Also, terms defined in general used dictionary are not to be excessively interpreted unless specifically defined

However, the present disclosure is not limited to embodiments disclosed below and may be implemented in various different forms. Some embodiments of the present disclosure are provided merely to fully inform those of ordinary skill in the technical field of the present disclosure of the scope of the present disclosure, and the present disclosure is only defined by the scope of the claims. Therefore, the definition should be made based on the content throughout the present disclosure.

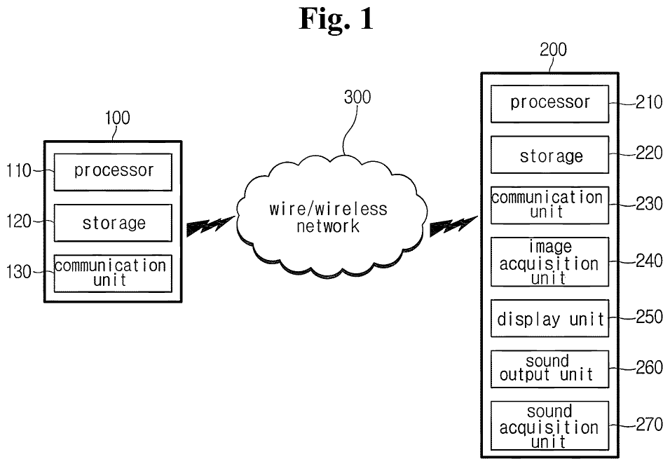

According to some embodiments of the present disclosure, at least one processor (hereinafter referred to as a “processor”) of a device may determine whether a user has dementia by using a dementia identification model. Specifically, the processor may obtain a score value by inputting, to the dementia identification model, a plurality of result data obtained through a plurality of tests. Furthermore, the processor may determine whether the user has dementia based on the score value. Hereinafter, a method of identifying dementia is described with reference to FIGS. 1 to 14.

FIG. 1 is a schematic diagram for explaining a system for identifying dementia according to some embodiments of the present disclosure.

Referring to FIG. 1, the system for identifying dementia may include a device 100 for identifying dementia and a user terminal 200 for a user requiring dementia identification. In addition, the device 100 and the user terminal 200 may be connected to communication over the wire/wireless network 300. However, the components constituting the system illustrated in FIG. 1 are not essential in implementing the system for identifying dementia, and thus more or fewer components than those listed above may be included.

The device 100 of the present disclosure may be paired with or connected to the user terminal 200 over the wire/wireless network 300, thereby transmitting/receiving predetermined data. In this case, data transmitted/received over the wire/wireless network 300 may be converted before transmission/reception. In this case, the “wire/wireless network” 300 collectively refers to a communication network supporting various communication standards or protocols for pairing and/or data transmission/reception between the device 100 and the user terminal 200. The wire/wireless network 300 includes all communication networks to be supported now or in the future according to the standard and may support all of one or more communication protocols for the same.

The device 100 for identifying dementia may include a processor 110, storage 120, and a communication unit 130. The components illustrated in FIG. 1 are not essential for implementing the device 100, and thus, the device 100 described in the present disclosure may include more or fewer components than those listed above.

Each component of the device 100 of the present disclosure may be integrated, added, or omitted according to the specifications of the device 100 that is actually implemented. That is, as needed, two or more components may be combined into one component or one component may be subdivided into two or more components. In addition, a function performed in each block is for describing an embodiment of the present disclosure, and the specific operation or device does not limit the scope of the present disclosure.

The device 100 described in the present disclosure may include any device that transmits and receives at least one of data, content, service, and application, but the present disclosure is not limited thereto.

The device 100 of the present disclosure includes, for example, any standing devices such as a server, a personal computer (PC), a microprocessor, a mainframe computer, a digital processor and a device controller; and any mobile devices (or handheld device) such as a smart phone, a tablet PC, and a notebook, but the present disclosure is not limited thereto.

In an embodiment of the present disclosure, the term “server” refers to a device or system that supplies data to or receives data from various types of user terminals, i.e., a client.

For example, a web server or portal server that provides a web page or a web content (or a web service), an advertising server that provides advertising data, a content server that provides content, an SNS server that provides a social network service (SNS), a service server provided by a manufacturer, a multichannel video programming distributor (MVPD) that provides video on demand (VoD) or a streaming service, a service server that provides a pay service, or the like may be included as a server.

In an embodiment of the present disclosure, the device 100 means a server according to context, but may mean a fixed device or a mobile device, or may be used in an all-inclusive sense unless specified otherwise.

The processor 110 may generally control the overall operation of the device 100 in addition to an operation related to an application program. The processor 110 may provide or process appropriate information or functions by processing signals, data, or information that is input or output through the components of the device 100 or driving an application program stored in the storage 120.

The processor 110 may control at least some of the components of the device 100 to drive an application program stored in the storage 120. Furthermore, the processor 110 may operate by combining at least two or more of the components included in the device 100 to drive the application program.

The processor 110 may include one or more cores, and may be any of a variety of commercial processors. For example, the processor 110 may include a central processing unit (CPU), general purpose graphics processing unit (GPUGP), and tensor processing unit (TPU) of the device, but the present disclosure is not limited thereto.

The processor 110 of the present disclosure may be configured as a dual processor or other multiprocessor architecture, but the present disclosure is not limited thereto.

The processor 110 may identify whether a user has dementia using the dementia identification model according to some embodiments of the present disclosure by reading a computer program stored in the storage 120.

The storage 120 may store data supporting various functions of the device 100. The storage 120 may store a plurality of application programs (or applications) driven in the device 100, and data, commands, and at least one program command for the operation of the device 100. At least some of these application programs may be downloaded from an external server through wireless communication. In addition, at least some of these application programs may exist in the device 100 from the time of shipment for basic functions of the device 100. The application program may be stored in the storage 120, installed in the device 100, and driven by the processor 110 to perform the operation (or function) of the device 100.

The storage 120 may store any type of information generated or determined by the processor 110 and any type of information received through the communication unit 130.

The storage 120 may include at least one type of storage medium of a flash memory type, a hard disk type, a solid state disk (SSD) type, a silicon disk drive (SDD) type, a multimedia card micro type, a card-type memory (e.g., SD memory and XD memory), random access memory (RAM), static random access memory (SRAM), read-only memory (ROM), electrically erasable programmable read-only memory (EEPROM), programmable read-only memory (PROM), magnetic memory, a magnetic disk, and an optical disk. The device 100 may be operated in relation to a web storage that performs a storage function of the storage 120 on the Internet.

The communication unit 130 may include one or more modules that enable wire/wireless communication between the device 100 and a wire/wireless communication system, between the device 100 and another device, or between the device 100 and an external server. In addition, the communication unit 130 may include one or more modules that connect the device 100 to one or more networks.

The communication unit 130 refers to a module for wired/wireless Internet connection, and may be built-in or external to the device 100. The communication unit 130 may be configured to transmit and receive wire/wireless signals.

The communication unit 130 may transmit/receive a radio signal with at least one of a base station, an external terminal, and a server on a mobile communication network constructed according to technical standards or communication methods for mobile communication (e.g., Global System for Mobile communication (GSM), Code Division Multi Access (CDMA), Code Division Multi Access 2000 (CDMA2000), Enhanced Voice-Data Optimized or Enhanced Voice-Data Only (EV-DO), Wideband CDMA (WCDMA), High Speed Downlink Packet Access (HSDPA), High Speed Uplink Packet Access (HSUPA), Long Term Evolution (LTE), Long Term Evolution-Advanced (LTE-A), etc.).

Examples of wireless Internet technology include Wireless LAN (WLAN), Wireless-Fidelity (Wi-Fi), Wireless Fidelity (Wi-Fi) Direct, Digital Living Network Alliance (DLNA), Wireless Broadband (WiBro), World Interoperability for Microwave Access (WiMAX), High Speed Downlink Packet Access (HSDPA), High Speed Uplink Packet Access (HSUPA), Long Term Evolution (LTE), and Long Term Evolution-Advanced (LTE-A). However, in a range including Internet technologies not listed above, the communication unit 130 may transmit/receive data according to at least one wireless Internet technology.

In addition, the communication unit 130 may be configured to transmit and receive signals through short range communication. The communication unit 130 may perform short range communication using at least one of Bluetooth™, Radio Frequency Identification (RFID), Infrared Data Association (IrDA), Ultra-Wideband (UWB), ZigBee, Near Field Communication (NFC), Wireless-Fidelity (Wi-Fi), Wi-Fi Direct and Wireless Universal Serial Bus (Wireless USB) technology. The communication unit 130 may support wireless communication through short range communication networks (wireless area networks). The short range communication networks may be wireless personal area networks.

The device 100 according to some embodiments of the present disclosure may be connected to the user terminal 200 and the wire/wireless network 300 through the communication unit 130.

In an embodiment of the present disclosure, the user terminal 200 may be paired with or connected to the device 100, in which the dementia identification model is stored, over the wire/wireless network 300, thereby transmitting/receiving and displaying predetermined data.

The user terminal 200 described in the present disclosure may include any device that transmits, receives, and displays at least one of data, content, service, and application. In addition, the user terminal 200 may be a terminal of a user who wants to check dementia, but the present disclosure is not limited thereto.

In an embodiment of the present disclosure, the user terminal 200 may include, for example, a mobile device such as a mobile phone, a smart phone, a tablet PC, or an ultrabook, but the present disclosure is not limited thereto. The user terminal 200 may include a standing device such as a Personal Computer (PC), a microprocessor, a mainframe computer, a digital processor, or a device controller.

The user terminal 200 includes a processor 210, storage 220, a communication unit 230, an image acquisition unit 240, a display unit 250, a sound output unit 260, and a sound acquisition unit 270. The components illustrated in FIG. 1 are not essential in implementing the user terminal 200, and thus, the user terminal 200 described in the present disclosure may have more or fewer components than those listed above.

Each component of the user terminal 200 of the present disclosure may be integrated, added, or omitted according to the specifications of the user terminal 200 that is actually implemented. That is, as needed, two or more components may be combined into one component, or one component may be subdivided into two or more components. In addition, the function performed in each block is for describing an embodiment of the present disclosure, and the specific operation or device does not limit the scope of the present disclosure.

The processor 210, storage 220, and communication unit 230 of the user terminal 200 are the same components as the processor 110, storage 120, and communication unit 130 of the device 100, and thus redundant descriptions thereof will be omitted, and differences between them are chiefly described below.

In an embodiment of the present disclosure, the processor 210 of the user terminal 200 may control the display unit 250 to display a screen for performing each of a plurality of tests in order to identify dementia. In this case, the plurality of tests may include at least one of a Stroop test, a calculation ability test, a memory test, a gaze test, and a mixed test, but is not limited thereto.

The Stroop test may refer to the effect that the reaction time for a given task varies according to attention, or a test conducted using such a phenomenon. The calculation ability test may mean a test that is performed in a way to provide an equation and select a correct answer according to the equation. The memory test may refer to a test that is performed in a way to memorize a plurality of objects displayed on a previous screen and select the plurality of objects on a screen that is now displayed. The gaze test may mean that a test is performed in a way to display a specific screen in order to obtain a movement of a user's gaze. The mixed test may mean a combination of a first test for obtaining first information related to a change in the user's gaze; and a second test for obtaining second information related to a user's voice. The tests are merely examples, and tests of the present disclosure are not limited thereto.

Since high processing speed and computational power are required to perform an operation using the dementia identification model, the dementia identification model may be stored only in the storage 120 of the device 100 and may not be stored in the storage 220 of the user terminal 200, but the present disclosure is not limited thereto.

The image acquisition unit 240 may include one or a plurality of cameras. That is, the user terminal 200 may be a device including one or plural cameras provided on at least one of a front part and rear part thereof.

The image acquisition unit 240 may process an image frame, such as a still image or a moving image, obtained by an image sensor. The processed image frame may be displayed on the display unit 250 or stored in the storage 220. The image acquisition unit 240 provided in the user terminal 200 may match a plurality of cameras to form a matrix structure. A plurality of image information having various angles or focuses may be input to the user terminal 200 through the cameras forming the matrix structure as described above.

The image acquisition unit 240 of the present disclosure may include a plurality of lenses arranged along at least one line. The plurality of lenses may be arranged in a matrix form. The plural lenses may be arranged in a matrix form. Such cameras may be called an array camera. When the image acquisition unit 240 is configured as an array camera, images may be captured in various ways using the plural lenses, and images of better quality may be obtained.

According to some embodiments of the present disclosure, the image acquisition unit 240 may obtain an image including an eye of a user, but the present disclosure is not limited thereto.

The display unit 250 may display (output) information processed by the user terminal 200. For example, the display unit 250 may display execution screen information of an application program driven in the user terminal 200, or user interface (UI) and graphic user interface (GUI) information according to the execution screen information.

The display unit 250 may include at least one of a liquid crystal display (LCD), a thin film transistor liquid crystal display (TFT LCD), an organic light-emitting diode (OLED), a flexible display, a 3D display, and an e-ink display, but the present disclosure is not limited thereto.

The sound output unit 260 may output audio data (or sound data, etc.) received from the communication unit 230 or stored in the storage 220. The sound output unit 260 may also output a sound signal related to a function performed by the user terminal 200.

The sound output unit 260 may include a receiver, a speaker, or a buzzer. That is, the sound output unit 260 may be implemented as a receiver or may be implemented in the form of a loudspeaker, but the present disclosure is not limited thereto.

According to some embodiments of the present disclosure, the sound output unit 260 may output a preset sound (e.g., a voice describing a task that needs to be now performed by a user) in interworking with any one of a plurality of tests being executed, but the present disclosure is not limited thereto.

The sound acquisition unit 270 may process an external sound signal as electrical sound data. The processed sound data may be used in various ways according to a function (or a running application program) being performed by the user terminal 200. Various noise removal algorithms for removing noise generated in a process of receiving an external sound signal may be implemented in the sound acquisition unit 270.

In an embodiment of the present disclosure, the processor 210 may obtain a recording file on which voices of a user have been recorded through the sound acquisition unit 270, in interworking with a specific screen being displayed, but the present disclosure is not limited thereto.

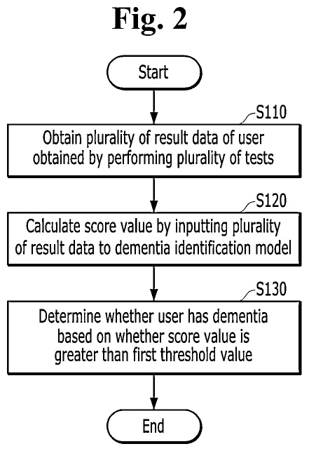

According to some embodiments of the present disclosure, a digital biomarker (e.g., a bio marker that is obtained from a digital device) for identifying dementia may be obtained by performing a plurality of tests in the user terminal. Furthermore, the processor 110 of the device 100 may identify whether a user has dementia by receiving the digital biomarker from the user terminal 200. This is described in detail with reference to FIG. 2.

FIG. 2 is a flowchart for describing an embodiment of a method of identifying whether a user has dementia according to some embodiments of the present disclosure. In relation to FIG. 2, contents that are redundant with those described in relation to FIG. 1 are not described again, and differences between FIGS. 1 and 2 are chiefly described below.

Referring to FIG. 2, the processor 110 of the device 100 may obtain a plurality of result data for a plurality of tests that has been performed by a user (S110).

Specifically, after screens for executing the plurality of tests are displayed, the processor 210 may obtain the plurality of result data from the user. The processor 210 may control the communication unit 230 to transmit the plurality of result data to the device 100. Furthermore, the processor 110 of the device 100 may obtain the plurality of result data by receiving the plurality of result data through the communication unit 230.

When the test is a Stroop test, the plurality of result data may include information on a total time that is taken for the Stroop test to be performed by a preset number of times, information on the number of times that an accurate answer has been determined while the user performs the Stroop test by a preset number of times, information on the number of times that an inaccurate answer has been determined while the user performs the Stroop test by a preset number of times, and information on at least one of response times taken while the user performs the Stroop test once.

When the test is a calculation ability test, the plurality of result data may include at least one of information on a total time that is taken for the calculation ability test to be performed by a preset number of times, information on the number of times that an accurate answer has been determined while the user performs the calculation ability test by a preset number of times, information on the number of times that an inaccurate answer has been determined while the user performs the calculation ability test by a preset number of times, and information on a response time taken while the user performs the calculation ability test once.

When the test is a memory test, the plurality of result data may include at least one of information on a total time that is taken for the memory test to be performed by a preset number of times, information on the number of times that an accurate answer has been determined while the user performs the memory test by a preset number of times, information on the number of times that an inaccurate answer has been determined while the user performs the memory test by a preset number of times, and information on a response time taken while the user performs the memory test once, but the present disclosure is not limited thereto. If gaze information of the user is also obtained while the memory test is performed, the plurality of result data may further include at least one of information on the order in which the user's gaze moves, information on whether the user's gaze for each of a plurality of objects displayed on a screen is maintained, and information on the time while the user's gaze for each of the plurality of objects has been maintained.

When the test is a gaze test, the plurality of result data may include at least one of information on the number of times that the user has correctly performed a preset gaze task, information on the number of times that the user has not correctly performed a preset gaze task, information on whether an eye of a user continues to stare at a specific point for a preset time, information on a response time that is taken for an eye of a user to move to a target while the user performs the gaze test, information on a moving speed of an eye of a user, and information on whether the user accurately stares at a target. In this case, the preset gaze task may be a task to indicate a target at which the user has to stare.

When the test is a mixed test, the plurality of result data may include at least one of first information related to a change in the user's gaze and second information obtained by analyzing a recording file on which voices of the user have been stored. In this case, the mixed test may mean a combination of a first test for obtaining the first information related to a change in the user's gaze and a second test for obtaining the second information related to a voice of the user. More specifically, the first information may include at least one of accuracy information that is calculated based on a distance in which an eye of a user has moved and a distance in which a target has moved, latency information that is calculated based on a time point at which a target starts to move and a time point at which the eye of the user starts to move, and speed information related to the speed at which the eye of the user has moved. Furthermore, the second information may include at least one of similarity information indicative of similarity between text data converted from a recording file through a voice recognition technology and original data, information on the speaking speed of the user, and response speed information that is calculated based on a time point at which a recording file has been obtained after the second test was performed.

In some embodiments of the present disclosure, the plurality of tests may be performed in a way to display at least one element along with the output of sound data and message data that describe a method of performing each of the plurality of tests. In this case, the at least one element may have a preset size or more. If a plurality of tests is performed as described above, the aged can easily understand and perform the contents of a test.

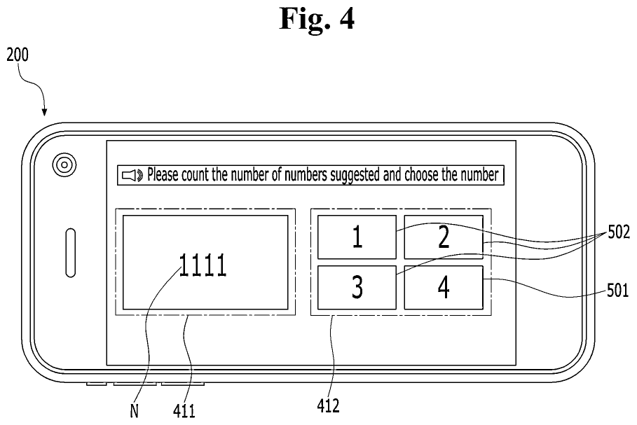

In an embodiment of the present disclosure, a method of displaying a screen for performing a plurality of tests is described in more detail with reference to FIGS. 4 to 13.

The processor 110 may calculate a score value by inputting the plurality of result data to the dementia identification model (S120). In this case, the score value may mean a value at which whether a user has dementia can be recognized based on a size of the value.

According to some embodiments of the present disclosure, the processor 110 may obtain user information (e.g., information on the age, sex, or address) of the user prior to step S110. Furthermore, the processor 110 may also calculate a score value by inputting, to the dementia identification model, at least some of the user information and the plurality of result data obtained after the plurality of tests is performed.

Specifically, the processor 110 may transmit a specific signal to the user terminal 200 so that a screen for obtaining the user information is displayed, prior to step S110. When the user information is received through the user terminal 200, the processor 210 of the user terminal 200 may control the communication unit 230 to transmit the user information to the device 100. In this case, the processor 110 of the device 100 may receive the user information through the communication unit 130. Furthermore, the processor 110 may calculate a score value by also inputting information on the age and sex of the user, that is, at least some of the user information, to the dementia identification model along with the plurality of result data that has been obtained by performing the plurality of tests.

If a score value is calculated by inputting, to the dementia identification model, at least some of the user information and the plurality of result data that has been obtained by performing the plurality of tests as described above, the accuracy of dementia identification can be further improved.

According to some embodiments of the present disclosure, a pre-learned dementia identification model may be stored in the storage 120 of the device 100.

The dementia identification model may be trained by a method of updating the weight of a neural network by backpropagating a difference value between label data labeled in learning data and prediction data output from the dementia identification model.

In an embodiment of the present disclosure, learning data may be obtained by performing, by a plurality of test users, each of a plurality of tests according to some embodiments of the present disclosure through their test devices. In this case, the learning data may include a plurality of result data that is obtained by performing, by the test user, the plurality of tests.

In an embodiment of the present disclosure, the test users may include a user classified as a patient with mild cognitive impairment (MCI), a user classified as an Alzheimer's patient, a user classified as normal, but the present disclosure is not limited thereto.

In an embodiment of the present disclosure, the test device may refer to a device where various test users perform tests when securing learning data. In this case, the test device may be a mobile device, such as a mobile phone, a smart phone, a tablet PC, or an ultrabook, similarly to the user terminal 200 used for dementia identification, but the present disclosure is not limited thereto.

In an embodiment of the present disclosure, the label data may be a score value capable of recognizing whether a patient is normal, is an Alzheimer's patient, and a patient with mild cognitive impairment, but the present disclosure is not limited thereto.

A dementia identification model may be composed of a set of interconnected computational units, which may generally be referred to as nodes. These nodes may also be referred to as neurons. The neural network may be configured to include at least one node. Nodes (or neurons) constituting the neural network may be interconnected by one or more links.

In the dementia identification model, one or more nodes connected through a link may relatively form a relationship between an input node and an output node. The concepts of an input node and an output node are relative, and any node in an output node relationship with respect to one node may be in an input node relationship in a relationship with another node, and vice versa. As described above, an input node-to-output node relationship may be created around a link. One output node may be connected to one input node through a link, and vice versa.

In the relation between the input node and the output node connected through one link, a value of data of the output node may be determined based on data that is input to the input node. In this case, the link interconnecting the input node and the output node may have a weight. The weight may be variable, and may be changed by a user or an algorithm so that the neural network performs a desired function.

For example, when one or more input nodes are connected to one output node by each link, the output node may determine an output node value based on values that are input to input nodes connected to the output node and based on a weight set in a link corresponding to each input node.

As described above, in the dementia identification model, one or more nodes may be interconnected through one or more links to form an input node and output node relationship in the neural network. The characteristics of the dementia identification model may be determined according to the number of nodes and links in the dementia identification model, a correlation between nodes and links, and a weight value assigned to each of the links.

The dementia identification model may consist of a set of one or more nodes. A subset of nodes constituting the dementia identification model may constitute a layer. Some of the nodes constituting the dementia identification model may configure one layer based on distances from an initial input node. For example, a set of nodes having a distance of n from the initial input node may constitute n layers. The distance from the initial input node may be defined by the minimum number of links that should be traversed to reach the corresponding node from the initial input node. However, the definition of such a layer is arbitrary for the purpose of explanation, and the order of the layer in the dementia identification model may be defined in a different way from that described above. For example, a layer of nodes may be defined by a distance from a final output node.

The initial input node may refer to one or more nodes to which data is directly input without going through a link in a relationship with other nodes among nodes in the neural network. Alternatively, in a relationship between nodes based on a link in the dementia identification model, it may mean nodes that do not have other input nodes connected by a link. Similarly, the final output node may refer to one or more nodes that do not have an output node in relation to other nodes among nodes in the neural network. In addition, a hidden node may refer to nodes constituting the neural network other than the first input node and the last output node.

In the dementia identification model according to some embodiments of the present disclosure, the number of nodes in the input layer may be greater than the number of nodes in the output layer, and the neural network may have a form in which the number of nodes decreases as it progresses from the input layer to the hidden layer. Furthermore, a plurality of result data may be input to each of the nodes of the input layer, but the present disclosure is not limited thereto.

According to some embodiments of the present disclosure, the dementia identification model may have a deep neural network structure.

A deep neural network (DNN) may refer to a neural network including a plurality of hidden layers in addition to an input layer and an output layer. The DNN may be used to identify the latent structures of data.

The DNN may include convolutional neural networks (CNNs), recurrent neural networks (RNNs), auto encoders, generative adversarial networks (GANs), and restricted Boltzmann machines (RBM), a deep belief network (DBN), a Q network, a U network, a Siamese network, and a generative adversarial network (GAN). These DNNs are only provided as examples, and the present disclosure is not limited thereto.

The dementia identification model of the present disclosure may be learned in a supervised learning manner, but the present disclosure is not limited thereto. The dementia identification model may be learned in at least one manner of unsupervised learning, semi supervised learning, or reinforcement learning.

Learning of the dementia identification model may be a process of applying knowledge for performing an operation of identifying dementia by the dementia identification model to a neural network.

The dementia identification model may be trained in a way that minimizes errors in output. Learning of the dementia identification model is a process of repeatedly inputting learning data (test result data for learning) into the dementia identification model, calculating errors of an output (score value predicted through the neural network) and target (score value used as label data) of the dementia identification model on the learning data, and updating the weight of each node of the dementia identification model by backpropagating the error of the dementia identification model from an output layer of the dementia identification model to an input layer in a direction of reducing the error.

A change amount of a connection weight of each node to be updated may be determined according to a learning rate. Calculation of the dementia identification model on the input data and backpropagation of errors may constitute a learning cycle (epoch). The learning rate may be differently applied depending on the number of repetitions of a learning cycle of the dementia identification model. For example, in an early stage of learning the dementia identification model, a high learning rate may be used to enable the dementia identification model to quickly obtain a certain level of performance, thereby increasing efficiency, and, in a late stage of learning the dementia identification model, accuracy may be increased by using a low learning rate.

In the learning of the dementia identification model, the learning data may be a subset of actual data (i.e., data to be processed using the learned dementia identification model), and thus, there may be a learning cycle wherein errors for learning data decrease but errors for real data increase. Overfitting is a phenomenon wherein errors on actual data increase due to over-learning on learning data as described above.

Overfitting may act as a cause of increasing errors in a machine learning algorithm. To prevent such overfitting, methods, such as increasing training data, regularization, and dropout that deactivate some of nodes in a network during a learning process, and the utilization of a batch normalization layer, may be applied.

According to some embodiments of the present disclosure, the dementia identification model may include a plurality of sub-models for receiving a plurality of result data, respectively. Furthermore, a score value may be calculated based on a plurality of sub-score values output by the plurality of sub-models, respectively.

For example, the score value may be an average value of the plurality of sub-score values that is output by the plurality of sub-models, respectively.

As another example, the weight of each of the plurality of sub-models may be added to each of the plurality of sub-score values output by the plurality of sub-models. Specifically, a weight may be preset in each of the plurality of sub-models based on the degree that each of the plurality of sub-models contributes to dementia identification. In this case, the sub-score value output by each of the plurality of sub-models may be multiplied by the weight that has been set in each of the plurality of sub-models. If a weight is added to each of a plurality of sub-score values as described above, the processor 110 may determine, as a score value, an average value of the plurality of sub-score values to which the weights have been added.

The aforementioned examples are merely embodiments of a method of calculating a score value according to some embodiments of the present disclosure, and the present disclosure is not limited thereto.

When the score value is calculated in step S120, the processor 110 according to some embodiments of the present disclosure may determine whether the user has dementia based on whether the score value is greater than a first threshold value. In this case, the first threshold value is a threshold value for identifying dementia, and may be previously stored in the storage 120, but the present disclosure is not limited thereto.

For example, when the score value is greater than the first threshold value, the processor 110 may determine that the user has dementia.

As another example, if the score value is greater than a second threshold value and is smaller than or equal to the first threshold value, the processor 110 may determine that the user has MCI. In this case, the second threshold value may be a value smaller than the first threshold value.

As another example, when the score value is smaller than or equal to the second threshold value, the processor 110 may determine that the user is normal.

In an embodiment of the present disclosure, the above examples are merely examples, and the present disclosure is not limited thereto.

According to some embodiments of the present disclosure, when determining that the user has MCI, the processor 110 of the device 100 may cause an application for improving cognitive power of the user to be executed in or downloaded to the user terminal. In this case, the application for improving cognitive power of the user may be an application that performs a function of improving the calculation ability, memory, etc. of the user through game, but the present disclosure is not limited thereto.

For example, the processor 110 of the device 100 may control the communication unit 130 to transmit a signal, including a download path of an application for improving cognitive power of a user, to the user terminal 200. In this case, when receiving the signal, the processor 210 of the user terminal 200 may control the communication unit 230 to download the application through the download path. However, if the application has already installed in the user terminal 200, the processor 210 of the user terminal 200 may execute the application without downloading the application. That is, when receiving the signal including the download path of the application, the processor 210 of the user terminal 200 may identify whether the application has already been installed, and may download the application only if the application has not been installed, but the present disclosure is not limited thereto.

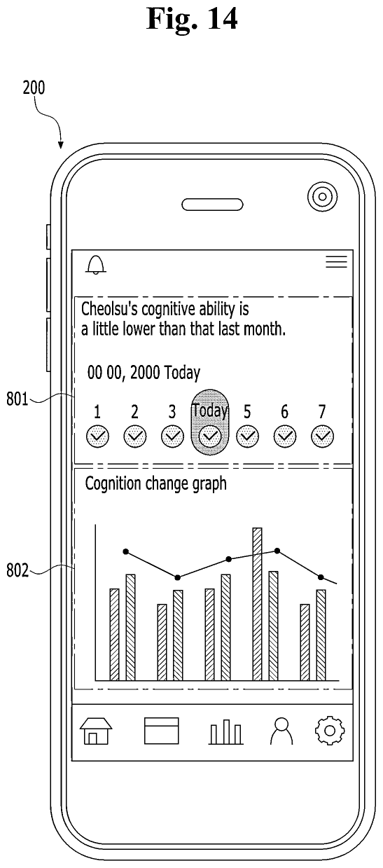

According to some embodiments of the present disclosure, if whether a user has dementia has been determined, the processor 110 may cause dementia identification result information to be output through a preset application of the user terminal of the user. In this case, the preset application may be a messenger application, but the present disclosure is not limited thereto. The dementia identification result information may be output to the user terminal through various applications. In an embodiment of the present disclosure, dementia identification result information that is output through a preset application is described in more detail with reference to FIG. 14.

According to some embodiments of the present disclosure, if it has been determined that a user has dementia, that is, if it is recognized that a score value is greater than the first threshold value, the processor 110 may cause the user terminal 200 to output hospital information that is generated based on information on the address of the user.

Specifically, prior to step S110, the processor 110 may obtain user information (e.g., information on the age, sex, or address) of the user. Furthermore, the processor 110 may extract information on hospitals that are located at a place close to a residence of the user based on the address information included in the user information. The processor 110 may search for a hospital in which treatment may be performed on a dementia patient, among hospitals located at the place close to the residence of the user. Furthermore, the processor 110 may control the communication unit 130 to transmit information on the retrieved hospital to the user terminal 200 so that the information on the retrieved hospital is displayed on the display unit 250 of the user terminal 200. In this case, after receiving the information on the retrieved hospital, the processor 210 of the user terminal 200 may control the display unit 250 to display the information on the retrieved hospital.

According to some embodiments of the present disclosure, a score value or dementia identification result information of a user may be used to calculate dementia-related insurance premium of the user. Accordingly, when the score value is calculated in step S120 or whether the user has dementia has been determined in step S130, the processor 110 may control the communication unit 130 to transmit the score value or the dementia identification result information of the user to an external server. In this case, the external server may be a server that is related to an insurance company, but the present disclosure is not limited thereto.

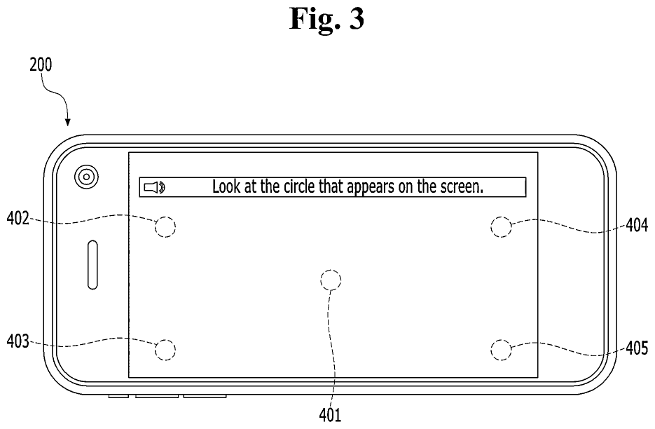

According to some embodiments of the present disclosure, the processor 110 of the device 100 may obtain geometric features of an eye of a user before a plurality of tests is performed or before a gaze test is performed. This is described in more detail with reference to FIG. 3.

FIG. 3 is a diagram for describing an embodiment of a method of obtaining the geometrical features of an eye of a user according to some embodiments of the present disclosure. In describing FIG. 3, the contents overlapping with those described above with reference to FIGS. 1 and 2 are not described again, and differences therebetween are mainly described below.

According to some embodiments of the present disclosure, before a plurality of tests is performed or a gaze test is performed, the user terminal 200 may display a specific screen for obtaining geometric features of an eye of a user.

Referring to FIG. 3, when a specific screen S is displayed in the user terminal 200, a preset object may be displayed in each of a plurality of regions 401, 402, 403, 404, and 405 for a preset time. In this case, the preset object may be a circular object having a diameter of 0.2 cm, but the present disclosure is not limited thereto.

For example, the processor 210 of the user terminal 200 may first control the display unit 250 such that the preset object is displayed in a first region 401 for a preset time (e.g., 3 to 4 seconds). Next, the processor 210 may control the display unit 250 to display the preset object in the second region 402 for a preset time (e.g., 3 to 4 seconds). In addition, the processor 210 may control the display unit 250 to sequentially display the preset object in each of the third region 403, the fourth region 404, and the fifth region 405 for a preset time (e.g., 3 to 4 seconds). In this case, when the preset object is displayed in any one of the plural regions 401, 402, 403, 404, and 405, the preset object may not be displayed in another region thereof. In this case, the order of the position in which the preset object is displayed is not limited to the aforementioned order.

When the preset object is displayed in each of the plural regions 401, 402, 403, 404, and 405 for a preset time, the processor 210 may obtain an image including an eye of a user through the image acquisition unit 240. In addition, geometrical features of the eye of the user may be obtained by analyzing the image. In this case, the geometrical features of the eye of the user are information necessary for accurately recognizing a change in the user's gaze, and may include the position of the center of the pupil, the size of the pupil of the eye, and the position of the eye of the user, but the present disclosure is not limited thereto.

For example, the processor 210 of the user terminal 200 may obtain geometrical features of an eye of a user by analyzing an image. In this case, a model for calculating the geometrical features of the eye of the user by analyzing an image may be stored in the storage 220 of the user terminal 200. The processor 210 may obtain the geometrical features of the eye of the user by inputting an image including an eye of a user to the model.

As another example, when an image is obtained through the image acquisition unit 240, the processor 210 of the user terminal 200 may control the communication unit 230 to transmit the image to the device 100. When the image is received through the communication unit 130, the processor 110 of the device 100 may analyze the image to obtain the geometrical features of the eye of the user. In this case, the model for calculating the geometrical features of the eye of the user by analyzing an image may be stored in the storage 120 of the device 100. The processor 110 may obtain the geometrical features of the eye of the user by inputting an image including an eye of a user to the model.

According to some embodiments of the present disclosure, the geometrical features of the eye of the user may be obtained based on a change in the position of the pupil of the user when the position at which a preset object is displayed is changed, but the present disclosure is not limited thereto. The geometrical features of the eye of the user may be obtained in various ways.

According to some embodiments of the present disclosure, the specific screen may include a message informing the user of a task to be performed through a currently displayed screen. For example, the message may include content to gaze at an object displayed on the specific screen, but the present disclosure is not limited thereto.

According to some embodiments of the present disclosure, a sound related to a message (e.g., a voice that explains contents included in the message) may be output through the sound output unit 260 in interworking with the message being displayed. In this manner, when a sound is output together with the message to allow the user to recognize a task to be performed by the user, it is possible to clearly understand what task the user should currently perform. Therefore, the possibility of performing a wrong operation by a simple mistake may be reduced.

In the case of obtaining the first information related to the change in the user's gaze after analyzing the geometrical features of the eye of the user while displaying the specific screen as in the aforementioned some embodiments, a change in the gaze may be accurately recognized without adding a separate component to the user terminal 200.

FIG. 4 is a diagram for describing an embodiment of a method of performing a Stroop test according to some embodiments of the present disclosure. In describing FIG. 4, the contents provided above with reference to FIGS. 1 and 2 will not be described again, and differences will be mainly described below.

Referring to FIG. 4, when the Stroop test is executed in a user terminal 200, a screen related to the Stroop test may be displayed on a screen of the user terminal 200.

Specifically, when a user of the user terminal 200 executes the Stroop test, the user terminal 200 may transmit a signal related to the execution of the Stroop test to the device 100. When the device 100 receives the signal related to the execution of the Stroop test, the device 100 may cause a screen related to the Stroop test to be displayed in the user terminal 200.

For example, the processor 110 of the device 100 may cause the user terminal 200 of a user to display the first number (e.g., four) of numeric text (e.g., “1”) in a first region 411 in interworking with a first button 501 being displayed, on which a first numeral (e.g., “4”) indicating a first number is displayed; and second buttons, on which second numerals (e.g., “1”, “2”, “3”) different from the first number are displayed, in a second region 412. In this case, the first region 411 may be a left region on the screen, and the second region 412 may be a right region on the screen, but the present disclosure is not limited thereto.

In accordance with some embodiments of the present disclosure, when a screen related to the Stroop test is displayed in the user terminal 200, a message related to the test content may be displayed in an arbitrary area. In this case, the user may recognize through the message what the given task is. In addition, the user terminal 200 may output a sound related to the message (e.g., a voice that explains contents included in the message) through the sound output unit, in interworking with the message being displayed on the screen through the display. If a user is made cognizant of a task that the user has to perform by outputting a sound along with a message as described above, there is a poor possibility that the user will select an incorrect answer.

The processor 110 may determine whether an answer is correct according to a first selection input of selecting any one of the first button 501 and the at least one second button 502.

Specifically, when the first selection input of selecting any one of the first button 501 and the at least one second button 502 is detected in the user terminal 200, the user terminal 200 may transmit information (e.g., information on which button is selected) on the first selection input to the device 100. In this case, the device 100 may determine whether the answer is correct based on the received information.

For example, the processor 110 of the device 100 may determine the answer as a correct answer when the first selection input is recognized as an input of selecting the first button 501, and may determine the answer as an incorrect answer when the first selection input is recognized as an input of selecting the at least one second button 502.

In accordance with some embodiments of the present disclosure, the Stroop test may be performed a preset first number of times while changing the first number of numeric texts and the numeric texts (N).

For example, when the first number of times is 2, the user terminal 200 may display four numeric texts (e.g., “1”) in the first area 411, and the processor 110 of the device 100 may determine whether the answer is correct according to the first selection input. Next, the user terminal 200 may display three different numeric texts (e.g., 2) in the first area 411, and the processor 110 of the device 100 may determine again whether the answer is correct according to the first selection input. The aforementioned example is merely an example, and the present disclosure is not limited thereto.

The Stroop test presented in FIG. 3 is to select the number of numeric texts presented in the first region 411, but the present disclosure is not limited thereto, and in the Stroop test, selecting the same numeric texts as numeric texts presented in the first region 411 may be presented as a task. In this case, the processor 110 of the device 100 may determine the answer as a correct answer when the first selection input is recognized as a selection button of selecting a button displaying the same numeric text as the numeric text displayed in the first region 411 of the user terminal 200, and may determine the answer as an incorrect answer when the first selection input is recognized as a selection input of selecting a button displaying a numeric text different from the numeric text displayed in the first region 411.

In accordance with some embodiments of the present disclosure, the processor 110 may perform a preliminary test such that the user may check the Stroop test before performing the Stroop test. In this case, since the preliminary test is performed in the same manner as the Stroop test, a detailed description thereof will be omitted.

The test result data obtained in the preliminary test may not be used when training the dementia identification model, and only the test result data obtained in the Stroop test may be used when training the dementia identification model. However, to increase the accuracy of the dementia identification of the dementia identification model, the test result data obtained in the preliminary test may also be used when training the dementia identification model.

The result data that is obtained through the Stroop test may include at least one of information on a total time that is taken for the Stroop test to be performed by a preset number of times, information on the number of times that an accurate answer has been determined, information on the number of times that an inaccurate answer has been determined, and information on a response time that is taken until a selection input to select any one of a plurality of buttons is received after a screen for the Stroop test is displayed.

However, in order to improve the accuracy of dementia identification of the dementia identification model, all of the information on the total time, the information on the number of times that an accurate answer has been determined, the information on the number of times that an inaccurate answer has been determined, and the information on the response time may be included in the result data.

At least one element that is included in a screen related to the Stroop test may include numeric text N and a plurality of buttons 501 and 502. In this case, the size of each of the numeric text N and the plurality of buttons 501 and 502 may be a preset size (e.g., a size which may be easily recognized by the aged) or more. If at least one element has a preset size or more as described above, the aged can easily perform a test.

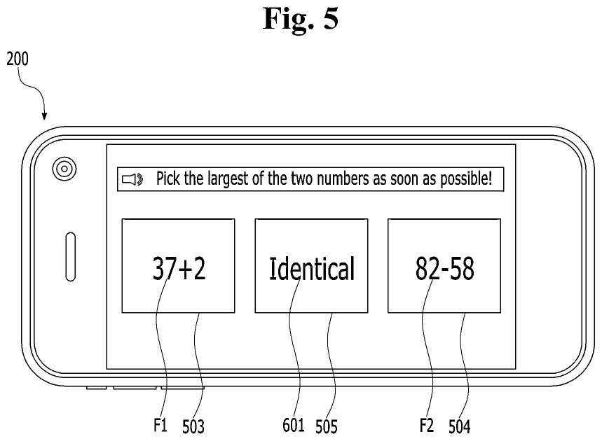

FIG. 5 is a diagram for describing an embodiment of a method of performing a calculation ability test according to some embodiments of the present disclosure. In describing FIG. 5, the contents provided above with reference to FIGS. 1 and 2 will not be described again, and differences will be mainly described below.

Referring to FIG. 5, when a calculation ability test is executed in the user terminal 200, a screen related to the calculation ability test may be displayed on a screen of the user terminal 200.

Specifically, when a user of the user terminal 200 executes the calculation ability test, the user terminal 200 may transmit a signal related to the execution of the calculation ability test to the device 100. When the device 100 receives the signal related to the execution of the calculation ability test, the device 100 may cause a screen related to the calculation ability test to be displayed in the user terminal 200.

For example, the processor 110 of the device 100 may cause the user terminal 200 of a user to display a third button 503 including a first equation F1, a fourth button 504 including a second equation F2, and a fifth button 505 including preset text 601.

The button 505 including the preset text 601 may be disposed between the button 503 including the first equation F1 and the button 504 including the second equation F2. If the button 505 including the preset text 601 is disposed at the aforementioned location, there may be a poor possibility that a user may select an inaccurate answer by mistake, but the present disclosure is not limited thereto.

The preset text 601 may be a text indicating that the result value of the first equation F1 and the result value of the second equation F2 are the same. For example, the preset text 601 “identical” may be included in the fifth button 505, but the present disclosure is not limited thereto.

The first equation F1 and the second equation F2 may include various types of equations. For example, the first equation F1 and the second equation F2 may be equations for adding, subtracting, multiplying, and dividing at least two numbers, but the present disclosure is not limited thereto.

In accordance with some embodiments of the present disclosure, when a screen related to the calculation ability test is displayed in the user terminal 200, a message related to the test content may be displayed in an arbitrary area. In this case, the user may recognize through the message what the given task is. In addition, the user terminal 200 may output a sound related to the message (e.g., a voice that explains contents included in the message) through the sound output unit, in interworking with the message being displayed on the screen through the display. If a user is made cognizant of a task that the user has to perform by outputting a sound along with a message as described above, there is a poor possibility that the user will check an incorrect answer by mistake.

The processor 110 may determine whether an answer is correct according to a selection input of selecting any one of the plurality of buttons 503, 504, 505.

Specifically, when the selection input of selecting any one of the plurality of buttons 503, 504, 505 is detected in the user terminal 200, the user terminal 200 may transmit information (e.g., information on which button is selected) on the selection input to the device 100. In this case, the device 100 may determine whether the answer is correct based on the received information.

Specifically, the processor 110 of the device 100 may determine whether the selection input is a correct answer based on a comparison result of a result value of the first equation and a result value of the second equation.

When the result value of the first equation F1 is greater than the result value of the second equation F2 as illustrated in FIG. 5, the processor 110 may determine that the answer is correct when recognizing that the button 503 is selected according to the selection input. In addition, the processor 110 may determine that the answer is incorrect when recognizing that any one of the buttons 504 and 505 other than the button 503 including the first equation F1 is selected according to the selection input. The reason for determining the correct answer as described above is that the task presented in the calculation ability test is to select a formula having a larger result value. Accordingly, if the task is changed, the correct answer may be changed. That is, if selecting a formula with a smaller result value is a task presented in the calculation ability test, the correct answer may be changed.

In accordance with some embodiments of the present disclosure, a calculation ability test may be performed a preset second number of times while changing the first equation F1 and the second equation F2.

In accordance with some embodiments of the present disclosure, the processor 110 may perform a preliminary test such that the user may check a test related to the calculation ability test before performing the calculation ability test. In this case, since the preliminary test is performed in the same manner as the aforementioned calculation ability test, a detailed description thereof will be omitted.

The test result data obtained through the preliminary test may not be used when training the dementia identification model, and only the test result data obtained through the calculation ability test may be used when training the dementia identification model. However, to increase the accuracy of the dementia identification of the dementia identification model, the test result data obtained in the preliminary test may also be used when training the dementia identification model.

The result data that is obtained through the calculation ability test may include at least one of information on a total time that is taken for the calculation ability test to be performed by a preset number of times, information on the number of times that an accurate answer has been determined, information on the number of times that an inaccurate answer has been determined, and information on a response time that is taken until a selection input to select any one of a plurality of buttons is received after a screen for the calculation ability test is displayed. However, in order to improve the accuracy of dementia identification, all of the information on the total time, the information on the number of times that an accurate answer has been determined, the information on the number of times that an inaccurate answer has been determined, and the information on the response time may be included in the result data.

At least one element that is included in a screen related to the calculation ability test may include the text 601, the plurality of equations F1 and F2, and the plurality of buttons 503 and 504. In this case, the size of each of the text 601, the plurality of equations F1 and F2, and the plurality of buttons 503 and 504 may be a preset size (e.g., a size which may be easily recognized by the aged) or more. If at least one element has a preset size or more as described above, the aged can easily perform a test.

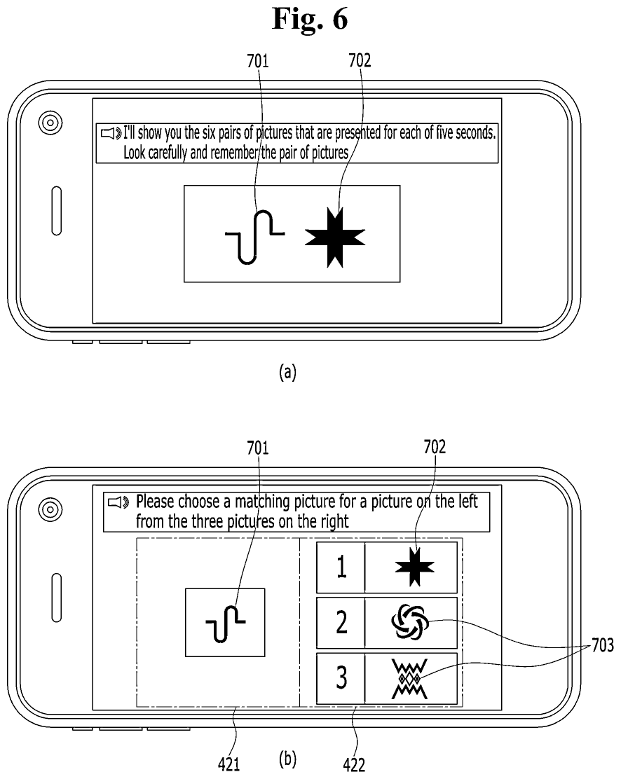

FIG. 6 is a diagram for describing an embodiment of a method of performing a memory test according to some embodiments of the present disclosure. In describing FIG. 6, the contents provided above with reference to FIGS. 1 and 2 will not be described again, and differences will be mainly described below.

When the memory test is executed in the user terminal 200, a screen related to the memory test may be displayed on the screen of the user terminal 200.

Specifically, when the user of the user terminal 200 executes the memory test, the user terminal 200 may transmit a signal, related to the execution of the memory test, to the device 100. When the device 100 receives a signal related to the execution of the memory test, the device 100 may cause a screen related to the memory test to be displayed in the user terminal 200.

For example, referring to FIG. 6(a), the processor 110 of the device 100 may cause the user terminal 200 of a user to display at least two objects 701 and 702 for a preset time (e.g., 10 seconds). In addition, referring to FIG. 6(b), the processor 110 may cause the user terminal 200 to display the first object 701 of the at least two objects in a third region 421 and to display the second object 702 of the at least two objects and at least one additional object 703 different from the at least two objects 701 and 702 in a fourth region 422.

Each of the at least two objects 701 and 702 of the present disclosure may have different forms and/or shapes. For example, the at least two objects may include a first object 701 having a first shape and a second object 702 having a second shape different from the first shape.

Each of the at least one additional object 703 of the present disclosure may have a form and/or shape different from each of the at least two objects 701 and 702. In addition, each of at least one additional object 703 may have a different form and/or shape.

The third region 421 may be located on the left side of the screen, and the fourth region 422 may be located on the right side of the screen, but the present disclosure is not limited thereto.

Referring back to FIG. 6(a), as the memory test is performed, a message indicating a task to be performed by the user may be displayed on a screen that is currently being displayed in the user terminal 200. For example, the message may include content to memorize at least two objects 701 and 702 that are currently being displayed on the screen.

Referring back to FIG. 6(b), a message indicating a task to be performed by the user may be displayed on a screen that is currently being displayed in the user terminal 200. For example, the message may include content to select the second object 702 included with the first object 701 displayed in the third region 421 on the screen displayed.

In addition, the user terminal 200 may output a sound related to a message (e.g., a voice that explains contents included in the message) through the sound output unit, in interworking with the message being displayed on the screen through the display. If a user is made cognizant of a task that the user has to perform by outputting the sound along with the message as described above, the user can clearly understand what task the user should perform.

The processor 110 may determine whether an answer is correct according to the selection input of selecting any one of the plural objects 702 and 703 displayed on the fourth region 422.

Specifically, when the selection input of selecting any one of the plural objects displayed on the fourth region 422 is detected, the user terminal 200 may transmit information (e.g., information on which object is selected) on the selection input to the device 100. In this case, the device 100 may determine whether the answer is correct based on the received information.

For example, the processor 110 may determine the answer as a correct answer when the selection input is recognized as an input of selecting the second object 702.

As another example, the processor 110 may determine the answer as an incorrect answer when the selection input is recognized as an input of selecting any one of the at least one additional object 703.

According to some embodiments of the present disclosure, the memory test may be performed by a preset number of times while the at least two objects 701 and 702 and the at least one additional object 703 are changed. In this case, when the plurality of objects 701, 702, and 703 is changed, all of the plurality of objects may be changed, and at least some of the plurality of objects 701, 702, and 703 may be changed, but the present disclosure is not limited thereto.

According to some embodiments of the present disclosure, when a selection input to select any one of the plurality of objects 702 and 703 displayed in the fourth region 422 is received, a selection input for all of the plurality of objects may be deactivated. In this case, the accuracy of identification of the dementia identification model can be improved because a case in which a user checks an inaccurate answer by mistake by performing a touch input unconsciously can be prevented.

In accordance with some embodiments of the present disclosure, the processor 110 may perform a preliminary test such that the user may check a memory test before performing the memory test. In this case, since the preliminary test is performed in the same manner as the aforementioned memory test, a detailed description thereof will be omitted.

The test result data obtained through the preliminary test may not be used when training the dementia identification model, and only the test result data obtained in the memory test may be used when training the dementia identification model. However, to increase the accuracy of the dementia identification of the dementia identification model, the test result data obtained in the preliminary test may also be used when training the dementia identification model.

The result data that is obtained through the memory test may include at least one of information on a total time that is taken for the memory test to be performed by a preset number of times, information on the number of times that an accurate answer has been determined, information on the number of times that an inaccurate answer has been determined, and information on a response time that is taken until a selection input to select any one of the objects 702 and 703 displayed in the fourth region 422 is received after a screen related to the memory test is displayed. However, in order to improve the accuracy of the dementia identification model, all of the information on the total time, the information on the number of times that an accurate answer has been determined, the information on the number of times that an inaccurate answer has been determined, and the information on the response time may be included in the result data.

At least one element that is included in a screen related to the memory test may include the plurality of objects 701, 702, and 703. In this case, the size of each of the plurality of objects 701, 702, and 703 may be a preset size (e.g., a size which may be easily recognized by the aged) or more. If at least one element has a preset size or more as described above, the aged can easily perform a test.

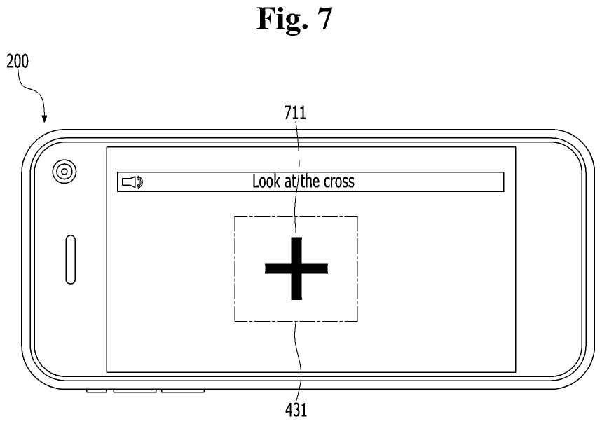

FIGS. 7 to 9 are diagrams for describing an embodiment of a method of performing a gaze test according to some embodiments of the present disclosure. In relation to FIGS. 7 to 9, contents that are redundant with those described in relation to FIGS. 1 and 2 are not described again, and differences between FIGS. 7 to 9 and FIGS. 1 and 2 are chiefly described below.

Referring to FIG. 7, before performing the gaze test, the processor 110 of the device 100 may cause a specific object 711 to be displayed in a central region 431 on a screen that is displayed in the user terminal 200.

For example, the processor 110 of the device 100 may generate a screen including the specific object 711 in the central region 431, and may transmit the screen to the user terminal 200. In this case, the user terminal 200 may display a screen in which the specific object 711 is included in the central region 431.

As another example, a screen in which the specific object 711 is included in the central region 431 may be stored in the storage 220 of the user terminal 200. When receiving a signal to display the screen stored in the storage 220 from the device 100 through the communication unit 230, the processor 210 of the user terminal 200 may control the display unit 250 to display the screen in the user terminal 200.

As another example, an image of the specific object 711 may be stored in the storage 220 of the user terminal 200. In this case, when the processor 110 of the device 100 transmits, to the user terminal 200, a signal to display a screen that includes the specific object 711 through the communication unit 130, the processor 210 of the user terminal 200 may generate a screen that includes the specific object 711 in the central region 431, and may display the screen.

However, the above examples are merely examples, and the present disclosure is not limited thereto.

In an embodiment of the present disclosure, the specific object 711 may be an object that induces a user's gaze to be located at the center of a screen that is displayed. For example, the first object 711 may be an object having a cross, but the specific object 711 is not limited thereto, and may have various forms or shapes.

In an embodiment of the present disclosure, the specific object 711 may be located in the central region 431. Accordingly, an eye of a user that stares at the specific object 711 may be located at the center of the screen, but the present disclosure is not limited thereto.

According to some embodiments of the present disclosure, a message including contents that provide notification of a task that needs to be performed by a user may be displayed on a screen that is displayed in the user terminal 200 through the screen that is now displayed. For example, the message may include contents indicating “Look at” the specific object 711 that is now displayed on a screen. Moreover, the user terminal 200 may output, through the sound output unit 260, a sound related to the message (e.g., a voice that explains contents included in the message) in interworking with the message being displayed on the screen through the display unit 250. If a user is made cognizant of a task that the user has to perform by outputting a sound along with a message as described above, the user can clearly understand what task the user should perform. Accordingly, there may be a poor possibility that the user may perform an erroneous task due to simple mistakes.

If the specific object 711 is displayed in the central region 431 of a screen, the processor 110 of the device 100 may confirm whether a preset condition is satisfied.

For example, the user terminal 200 may obtain an image including an eye of a user who uses the user terminal through the image acquisition unit 240, in interworking with the specific object 711 being displayed (performing a first task) in the central region 431 of a screen. The user terminal 200 may confirm whether a preset condition is satisfied by analyzing the image. Furthermore, when the user terminal 200 recognizes that the preset condition has been satisfied, the processor 210 of the user terminal 200 may transmit a signal, indicating that the preset condition has been satisfied, to the device 100 through the communication unit 230. In this case, the device 100 may recognize that the preset condition has been satisfied.

As another example, the user terminal 200 may obtain an image including an eye of a user who uses the user terminal through the image acquisition unit 240, in interworking with the specific object 711 being displayed (performing a first task) in the central region 431 of a screen. The user terminal 200 may control the communication unit 230 to transmit the obtained image to the device 100. When receiving the image including an eye of a user through the communication unit 130, the processor 110 of the device 100 may confirm whether a preset condition is satisfied by analyzing the image.

However, the above examples are merely examples, and the present disclosure is not limited thereto.

The preset condition may be satisfied if it is recognized that a user stares at the specific object 711 for a preset time by analyzing an image that is obtained while the specific object 711 is displayed.

When recognizing that the preset condition has been satisfied, the device 100 may cause at least one object to be displayed on a screen of the user terminal 200 instead of the specific object 711.

If the preset condition is satisfied in the state in which a screen including the specific object 711 illustrated in FIG. 7 has been displayed, the following task may be performed.

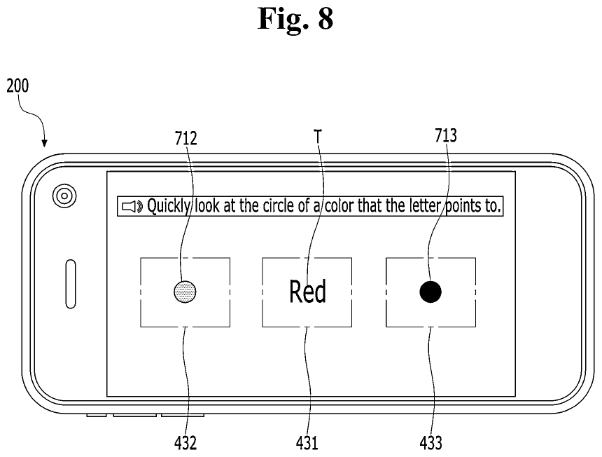

Referring to FIG. 8, when recognizing that a preset condition has been satisfied, the device 100 may cause the user terminal 200 to display a screen including a first object 712, a second object 713, and text T. In this case, the user terminal 200 may display the first object 712 and the second object 713 in regions of the central region 431 on both sides thereof, respectively, in interworking with the text T being displayed in the central region 431 instead of the specific object 711. That is, at least one object may include the text T that is displayed in the central region 431, and the first object 712 and the second object 713 that are displayed in the regions of the central region 431 on both sides thereof, respectively. In this case, the screen including the first object 712, the second object 713, and the text T may be displayed for 2000 ms, but the present disclosure is not limited thereto.

The first object 712 and the second object 713 may have the same shape (e.g., a circle having a diameter of 0.2 cm), but may have different colors only. In this case, any one of the first object 712 and the second object 713 may have a color that is meant by the text T, and the other thereof may have a color different from that is meant by the text T, but the present disclosure is not limited thereto. The first object 712 and the second object 713 may have different shapes, and colors thereof may be different from each other.

The text T may be a word that means a color or shape, but the meaning of the text T is not limited thereto.

If the meaning of the text T is related to color, the color of the text T itself may be the same as or different from the color that the text T means. However, the present invention is not limited thereto.

According to some embodiments of the present disclosure, the message of a content indicating what a preset gaze task that a user should perform is displayed may be displayed on a screen displayed in the user terminal 200. In this case, the preset gaze task may indicate which object a user should stare at.

According to some embodiments, the preset gaze task may be a task for staring at an object related to the meaning of the text T among the first object 712 and the second object 713. In this case, the meaning of the text T may be related to shape or color, but the meaning of the text T is not limited thereto.

For example, the text T may mean red, the first object 712 may have red, and the second object 713 may have blue. In addition, the message displayed on the screen may include the content to stare at the object of the color indicated by the text T. In this case, when a user stares at the first object 712, it may be considered that the user performs the preset task correctly.

As another example, although not illustrated in the drawing, the text T may mean a circle, the first object 712 may have a circular shape, and the second object 713 may have a rectangular shape. In addition, a message displayed on the screen may include the content to stare at an object having a shape indicated by the text T. In this case, when a user stares at the first object 712, it may be considered that the user performs the preset task correctly.

According to some other embodiments, the preset gaze task may be a task to stare at an object unrelated to the meaning of the text T among the first object 712 and the second object 713. In this case, the meaning of the text T may be related to shape or color, but the meaning of the text T is not limited thereto.

For example, the text T may mean red, the first object 712 may have red, and the second object 713 may have blue. In addition, the message displayed on the screen may include a content to stare at an object that does not have a color indicated by the text T. In this case, if a user stares at the second object 713, it may be considered that the user performs the preset task correctly.

As another example, the text T may mean a circle, the first object 712 may have a circular shape, and the second object 713 may have a rectangular shape. In addition, the message displayed on the screen may include a content to stare at an object that does not have a shape indicated by the text T. In this case, if a user stares at the second object 713, it may be considered that the user performs the preset task correctly.

According to some other embodiments, the preset gaze task may be a task to stare at an object related to the color of the text T itself among the first object 712 and the second object 713. In this case, the color of the text T itself may be different from the meaning of the text T, or may be the same as the meaning of the text T.

For example, the text T may mean red, the text T may be red, the first object 712 may have red, and the second object 713 may have blue. In addition, the message displayed on the screen may include a content to stare at an object having the color of the text T. In this case, if a user stares at the first object 712, it may be considered that the user performs the preset task correctly.

As another example, the text T may mean red, the color of the text T may be blue different from the meaning of the text T, the first object 712 may have red, and the second object 713 may have blue. In addition, the message displayed on the screen may include a content to stare at an object having the color of the text T. In this case, if a user stares at the second object 713, it may be considered that the user performs the preset task correctly.

According to some other embodiments, the preset gaze task may be a task to stare at an object unrelated to the color of the text T itself among the first object 712 and the second object 713. In this case, the color of the text T itself may be different from the meaning of the text T, or may be the same as the meaning of the text T.

For example, the text T may mean red, the text T may be red, the first object 712 may have red, and the second object 713 may have blue. In addition, the message displayed on the screen may include a content to stare at an object that does not have the color of the text T. In this case, if a user stares at the second object 713, it may be considered that the user performs the preset task correctly.

As another example, the text T may mean red, the color of the text T may be blue different from the meaning of the text T, the first object 712 may have red, and the second object 713 may have blue. In addition, the message displayed on the screen may include a content to stare at an object that does not have the color of the text T. In this case, if a user stares at the first object 712, it may be considered that the user performs the preset task correctly.

When a preset condition is satisfied in the state in which a screen including the specific object 711 illustrated in FIG. 7 has been displayed, the following task different from the task described with reference to FIG. 8 may be performed.

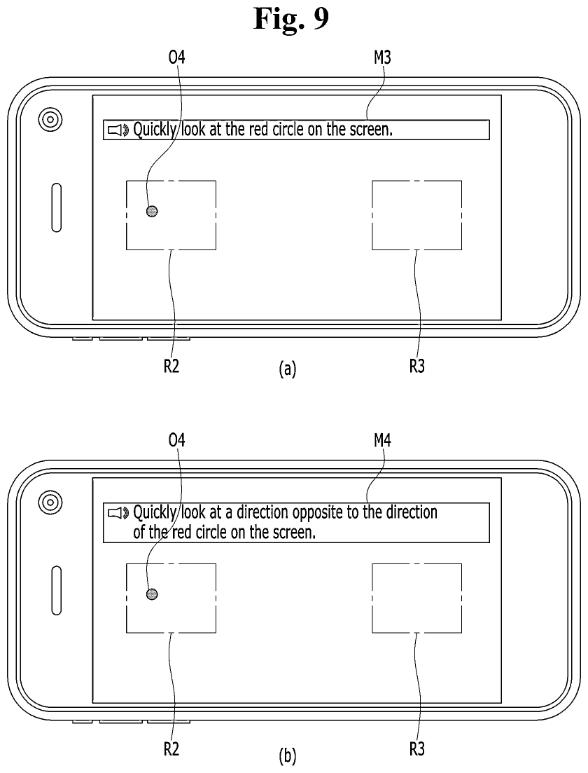

Referring to FIG. 9, the processor 110 of the device 100 may cause the user terminal 200 to display a gaze induction object in any one of the second region R2 and third region R3 that are different from the central region 431. In this case, the user terminal 200 may display a gaze induction object O4 in the second region R2 or the third region R3 different from the central region 431, instead of displaying the first object. That is, at least one object may include the gaze induction object O4 that is displayed in any one of the second region R2 and third region R3 that are different from the first region 431. The second region R2 and the third region R3 in FIG. 9 are regions different from the second region 402 and the third region 403 in FIG. 3.

More specifically, referring to FIGS. 9(a) and 9(b), when a preset condition is satisfied, a screen that is displayed in the user terminal 200 may include the gaze induction object O4 that is displayed in any one region R2 of the second region R2 and third region R3 that are different from the central region 431 in FIG. 7. In this case, any one of the second region R2 and the third region R3 may be randomly selected. The screen including the gaze induction object O4 may be displayed for 2000 ms, but the present disclosure is not limited thereto.

The gaze induction object O4 may be an object having a preset shape (e.g., a circle having a diameter of 0.2 cm) and a preset color (e.g., red).

The central region 431 may be located between the second region R2 and the third region R3. That is, the second region R2 and the third region R3 may be located on both sides of the central region 431 that is located in the center of a screen, but the present disclosure is not limited thereto.

According to some embodiments of the present disclosure, messages M3 and M4 including contents that provide notification of what preset gaze task should a user perform may be displayed on a screen that is displayed in the user terminal 200. In this case, the preset gaze task may indicate what object should the user stare at.

Referring to FIG. 9(a), the preset gaze task may be a task to stare at the gaze induction object O4. In addition, a message M3 displayed on the screen may include a content to quickly stare at the gaze induction object O4. In this case, if a user stares at the gaze induction object O4, it may be considered that the user performs the preset task correctly.

Referring to FIG. 9(b), the preset gaze task may be a task to stare at in an opposite direction to the direction in which the gaze induction object O4 is located. In addition, the message M4 displayed on the screen may include a content to quickly stare at the gaze induction object O4. In this case, if a user stares at any point in an opposite direction to the gaze induction object O4, it may be considered that the user performs the preset task correctly.

The user terminal 200 may output a sound related to the message M3 or M4 (e.g., a voice that explains contents included in the message M3 or M4) through the sound output unit 260 in interworking with the message M3 or M4 being displayed on the screen through the display unit 250. In this manner, when a user is recognized with a preset gaze task that the user should perform with a sound together with the message M3 or M4, the user can clearly understand what the preset gaze task that the user should currently perform is. Therefore, the possibility of performing a wrong gaze task by simple mistake may be lowered.

As a result, the preset gaze task may include at least one of a task to stare at a gaze induction object and a task to stare at in a direction opposite to the direction in which a gaze induction object is positioned. In addition, the message M3 or M4 displayed on the screen may include information on what the preset gaze task is.

The user terminal 200 may also output, through the sound output unit 260, a sound related to a message (e.g., a voice that explains contents included in the message), in interworking with the message being displayed on the screen through the display unit 250. If a user is made cognizant of a preset gaze task that needs to be performed by the user through a sound along with a message as described above, the user can clearly understand what preset gaze task the user should now perform. Accordingly, there may be a poor possibility that the user will perform an erroneous gaze task due to simple mistakes.

The processor 110 of the device 100 may cause a gaze test to be performed by a preset number of times (e.g., 5 times). That is, after the processor 110 causes the user terminal 200 to display the screen illustrated in FIG. 7, when recognizing that the user stares at the center of the screen, the processor 110 may perform a task that causes the screen illustrated in FIG. 8 or 9 to be displayed in the user terminal 200 by a preset number of times.

As in an embodiment of the present disclosure, if a user's gaze is made located at the center of a screen before a gaze test is performed, whether a user has dementia can be accurately identified although a separate component is not added to the user terminal 200 that is used by the user.

If an ordinary person, not a dementia patient, does not stare at the center of a screen and has to stare at an object that is displayed on the left or right of the screen, the ordinary person, not a dementia patient, may be determined to have dementia because the ordinary person has not quickly moved his or her gaze. Accordingly, the accuracy of dementia identification can be improved only if a user stares at the center of a screen and stares at an object that is displayed on the left or right of the screen. Accordingly, according to an embodiment of the present disclosure, a screen including a specific object at the center of the screen may be displayed so that a user can stare at the center of the screen before a screen including text and at least one object is displayed.

According to some embodiments of the present disclosure, the processor 110 of the device 100 may obtain gaze information related to a user while a gaze test is performed by a preset number of times. In this case, the gaze information may be used as a digital biomarker (e.g., a bio marker that is obtained from a digital device) for identifying dementia.

For example, the processor 210 of the user terminal 200 may obtain an image including an eye of a user while a gaze test is performed by a preset number of times. In this case, while the gaze test is performed by a preset number of times, the processor 110 of the device 100 may receive the image including an eye of a user through the communication unit 130 from the user terminal 200. When receiving the image, the processor 110 may generate gaze information by analyzing the image.

As another example, while a gaze test is performed by a preset number of times, the processor 210 of the user terminal 200 may obtain an image including an eye of a user. The processor 210 may obtain gaze information by analyzing the obtained image. Furthermore, the processor 210 may control the communication unit 230 to transmit the obtained gaze information to the device 100. In this case, the processor 110 of the device 100 may receive the gaze information through the communication unit 130 from the user terminal 200.

Result data that is obtained through the gaze test may include gaze information. In this case, the gaze information may include at least one of information on the number of times that a user has correctly performed a preset gaze task, information on the number of times that a user has not correctly performed a preset gaze task, information on whether an eye of a user continues to stare at a specific point for a preset time, information (e.g., information on the time for which a response from a user has been delayed) on the time that is taken from a time point at which a screen including at least one object is displayed to a time point at which an eye of a user has moved to any one of at least one object, information on a moving speed of an eye of a user, and information on whether a user accurately stares at a point related to a preset gaze task. However, in order to improve the accuracy of the dementia identification model, all the types of information included in the gaze information may be included in the result data.

According to some embodiments of the present disclosure, before a gaze test is performed, the processor 110 may perform a preliminary test so that a user can check a preset gaze task. In this case, the preliminary test is performed in the same manner as the gaze test, and a detailed description thereof is omitted.

Gaze information that is obtained through the preliminary test may not be used to identify whether a user has dementia by using the dementia identification model, but the present disclosure is not limited thereto. In order to increase the accuracy of dementia identification of the dementia identification model, gaze information obtained through the preliminary test may also be input to the dementia identification model.

At least one element that is included in a screen related to a gaze test may include at least one object (e.g., the specific object 711, the text T, and the plurality of objects 712 and 713 in FIG. 8 or the gaze induction object O4 in FIG. 9). In this case, the size of the at least one object may be a preset size (e.g., a size which may be easily recognized by the aged) or more. If at least one element has a preset size or more as described above, the aged can easily perform a test.

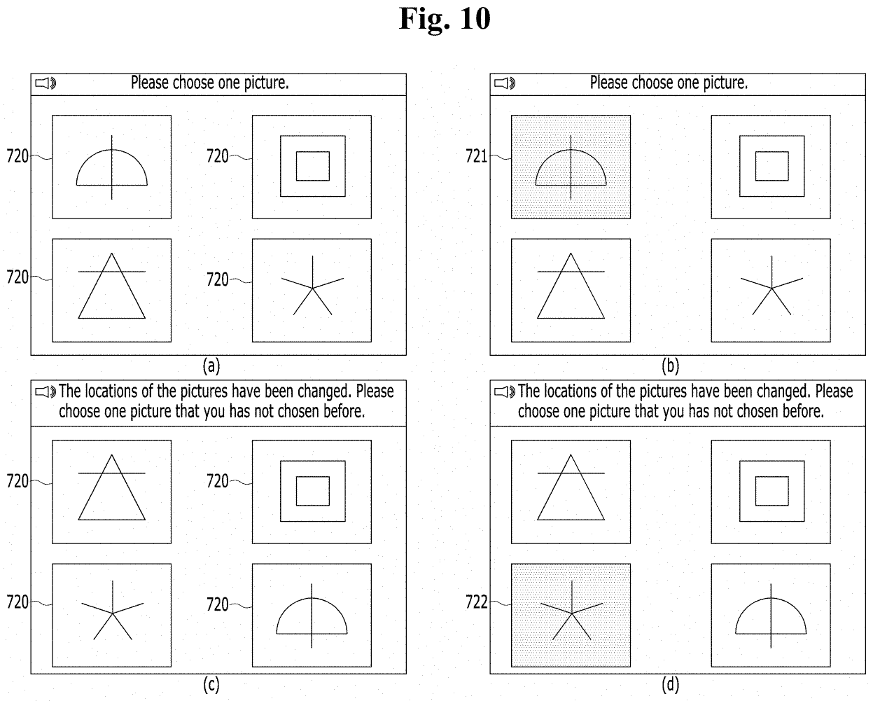

FIG. 10 is a diagram for describing another embodiment of a method of performing a memory test according to some embodiments of the present disclosure. In relation to FIG. 10, contents that are redundant with those described in relation to FIGS. 1 and 2 are not described again, and differences between FIG. 10 and FIGS. 1 and 2 are chiefly described below.

Referring to FIG. 10(a), when a memory test is performed, the processor 110 of the device 100 may cause an N-th screen including a plurality of objects 720 to be displayed in the user terminal 200. In this case, N may be a natural number equal to or greater than 1. Furthermore, the plurality of objects O may be objects at least one of a shape and form of each of which is different.

For example, the processor 110 of the device 100 may generate an N-th screen including the plurality of objects 720, and may transmit the N-th screen to the user terminal 200. In this case, the processor 210 of the user terminal 200 may control the display unit 250 to display the N-th screen including the plurality of objects 720.

As another example, a plurality of screens in which the plurality of objects 720 has been disposed at different locations may be stored in the storage 220 of the user terminal 200. When receiving a signal to display a screen including the plurality of objects 720 from the device 100 through the communication unit 230, the processor 210 of the user terminal 200 may display, as an N-th screen, any one of the plurality of screens stored in the storage 220.

As another example, an image of the plurality of objects 720 may be stored in the storage 220 of the user terminal 200. When receiving a signal to display a screen including the plurality of objects 720 from the device through the communication unit 230, the processor 210 of the user terminal 200 may generate an N-th screen including the plurality of objects 720, and may display the N-th screen.

The N-th screen may include a message including contents that provide notification of a task that needs to be performed by a user through a screen that is now displayed. For example, if the N-th screen is a primary screen that is displayed for the first time, the message may include contents indicating that any one of a plurality of objects included in the primary screen should be selected, but the present disclosure is not limited thereto.

According to some embodiments of the present disclosure, a sound related to a message (e.g., a voice that explains contents included in the message) may also be output in interworking with the message being displayed through the sound output unit 260. If a user is made cognizant of a task that needs to be performed by the user by outputting a sound along with a message as described above, the user can clearly understand a task that the user should now perform. Accordingly, there may be a poor possibility that the user may perform an erroneous task due to simple mistakes.

Referring to FIG. 10(b), the processor 210 of the user terminal 200 may receive an N-th selection input to select any one object 721 of the plurality of objects 720.

In an embodiment of the present disclosure, when receiving an N-th selection input, the processor 210 may deactivate an additional selection input for an N-th screen. In this case, the additional selection input may mean a selection input that is additionally detected after a selection input to first select any one object is detected in the state in which the N-th screen has been displayed.

If an additional selection input for an N-th screen is deactivated as described above, an error which occurs when a user additionally touches an arbitrary region on the N-th screen by mistake can be reduced.

The processor 210 of the user terminal 200 may control the display unit 250 to display the object 721, selected among the plurality of objects 720 through an N-th selection input, by incorporating a preset effect into the object 721. For example, only the object 721 selected among the plurality of objects 720 through the N-th selection input may be highlighted in a color different from those of other objects, but the present disclosure is not limited thereto.

Referring to FIG. 10(c), when detecting an N-th selection input to select any one 721 of the plurality of objects 720, the processor 110 of the device 100 may cause the user terminal 200 to display an (N+1)-th screen in which the plurality of objects has been rearranged.

For example, when detecting the N-th selection input, the processor 210 of the user terminal 200 may control the communication unit 230 to transmit an N-th signal, indicating that the N-th selection input has been detected, to the device 100. In this case, the N-th signal may include information indicating which one of the plurality of objects 720 has been selected. When receiving the N-th signal, the processor 110 of the device 100 may generate the (N+1)-th screen in which the plurality of objects has been rearranged, and may control the communication unit 130 to transmit the (N+1)-th screen to the user terminal 200. When receiving the (N+1)-th screen through the communication unit 230, the processor 210 of the user terminal 200 may control the display unit 250 to display the (N+1)-th screen.

As another example, a plurality of screens in which the plurality of objects 720 has been disposed at different locations may be stored in the storage 220 of the user terminal 200. When detecting an N-th selection input, the user terminal 200 may transmit an N-th signal, indicating that the N-th selection input has been detected, to the device 100. In this case, the N-th signal may include information indicating which one of the plurality of objects 720 has been selected. Furthermore, the processor 210 may select a screen that has not been displayed before, among the plurality of screens stored in the storage 220, and may control the display unit 250 to display the selected screen. If a screen that has not been displayed before is displayed, the screen may seem like that a screen in which the plurality of objects 720 has been rearranged is displayed.

As another example, an image of the plurality of objects 720 may be stored in the storage 220 of the user terminal 200. When detecting an N-th selection input, the user terminal 200 may transmit an N-th signal, indicating that the N-th selection input is detected, to the device 100. In this case, the N-th signal may include information indicating which one of the plurality of objects 720 has been selected. The processor 210 of the user terminal 200 may generate an (N+1)-th screen in which a plurality of objects has been rearranged so that the plurality of objects is displayed at locations different from those of a plurality of objects displayed on an N-th screen. Furthermore, the processor 210 may control the display unit 250 to display the (N+1)-th screen.

Referring to FIGS. 10(a) and 10(c), the locations of the plurality of objects 720 included in the N-th screen may be different from those of the plurality of objects 720 included in the (N+1)-th screen, respectively.

That is, the processor 110 may rearrange the plurality of objects included in the (N+1)-th screen by randomly changing the locations of the plurality of objects 720 included in the N-th screen.

The (N+1)-th screen may include a message including contents that provide notification of a task that needs to be performed by a user through a screen that is now displayed. For example, the message may include contents to select any one object not selected in a previous screen, among the plurality of objects included in the (N+1)-th screen, but the present disclosure is not limited thereto.

According to some embodiments of the present disclosure, a sound related to a message (e.g., a voice that explains contents included in the message) may be output through the sound output unit 260 in interworking with the message being displayed. If a user is made cognizant of a task that needs to be performed by the user by outputting a sound along with a message as described above, the user can clearly understand a task that the user should now perform. Accordingly, there may be a poor possibility that the user may perform an erroneous task due to simple mistakes.

Referring to FIG. 10(d), when detecting an (N+1)-th selection input to select any one of the plurality of objects displayed on an (N+1)-th screen, the processor 110 of the device 100 may determine whether the (N+1)-th selection input is an accurate answer, based on whether the object selected through the (N+1)-th selection input and at least one object selected through at least one previous selection input are identical with each other.

For example, when N is 1, the processor 110 may recognize whether an object selected through a second selection input is the same as an object selected through a first selection input that is a previous selection input. The processor 110 may determine that the second selection input is an incorrect answer when the object selected through the first selection input is the same as the object selected through the second selection input, and may determine that the second selection input is a correct answer when the object selected through the first selection input is different from the object selected through the second selection input.

As another example, when N is 2, the processor 110 may recognize whether an object selected through a third selection input is the same as a plurality of objects selected through a previous first selection input and a previous second selection input. The processor 110 may determine that the third selection input is a correct answer when both the object selected through the first selection input and the object selected through the second selection input are different from the object selected through the third selection input, and may determine that the third selection input is an incorrect answer when any one of the object selected through the first selection input and the object selected through the second selection input is the same as the object selected through the third selection input.

In an embodiment of the present disclosure, when receiving an (N+1)-th selection input, the processor 210 may deactivate an additional selection input for an (N+1)-th screen. In this case, the additional selection input may mean a selection input additionally detected after a selection input to select any one object is detected for the first time in the state in which the (N+1)-th screen has been displayed.

As described above, when the additional selection input for the (N+1)-th screen is inactivated, an error occurring when a user additionally touches an arbitrary area on the (N+1)-th screen by mistake may be reduced.

The processor 210 of the user terminal 200 may control the display unit 250 to display an object 722 selected among the plurality of objects 720 through the (N+1)-th selection input by incorporating a preset effect into the object 722. For example, only the object 722, selected among the plurality of objects 720 through the (N+1)-th selection input, may be highlighted in a color different from that of other objects, but the present disclosure is not limited thereto.

According to some embodiments of the present disclosure, the processor 110 of the device 100 may perform a memory test by a preset number of times. In this case, if the memory test is further performed M (M is a natural number equal to or greater than 1) times, M may be added to N. That is, when detecting a selection input to select any one of a plurality of objects included in a screen that is displayed in the user terminal 200, the processor 110 may cause the locations of the plurality of objects included in the screen displayed in the user terminal 200 to continue to be changed by a preset number of times. Furthermore, the processor 110 may determine whether a current selection input is an accurate answer, based on whether at least one object selected through at least one previous selection input and an object selected through the current selection input are identical with each other.

According to some embodiments of the present disclosure, the processor 110 may perform a preliminary test so that a user can check how the user should perform a memory test before performing the memory test. In this case, the preliminary test is performed in the same manner as the memory test, and a detailed description thereof is omitted.

Data that is obtained in the preliminary test may not be used as a digital biomarker that is input to the dementia identification model in order to identify whether a user has dementia, but the present disclosure is not limited thereto. Data obtained in a preliminary test may also be used as a digital biomarker.

According to some embodiments of the present disclosure, the processor 110 of the device 100 may obtain gaze information based on an image including an eye of a user, in interworking with a memory test being performed. In this case, the gaze information may include at least one of information on the order of the user's gaze being moved, information on whether the user's gaze for each of a plurality of objects displayed on a screen is maintained, and information on the time while the user's gaze for each of the plurality of objects has been maintained.

The result data that is obtained through the memory test may include gaze information, information on the number of times that an accurate answer has been determined, information on the number of times that an inaccurate answer has been determined, and information on a response time for the time that is taken until an N-th selection input or an (N+1)-th selection input to be received in the state in which an N-th screen or an (N+1)-th screen has been displayed. However, in order to improve the accuracy of the dementia identification model, all of the gaze information, the information on the number of times that an accurate answer has been determined, the information on the number of times that an inaccurate answer has been determined, and the information on the response time may be included in the test result data.

In FIG. 10, at least one element included in the screen related to the memory test may include the plurality of objects 720. In this case, each of the plurality of objects 720 may have a preset size or more. If at least one element has a preset size (e.g., a size which may be easily recognized by the aged) or more as described above, the aged can easily perform a test.

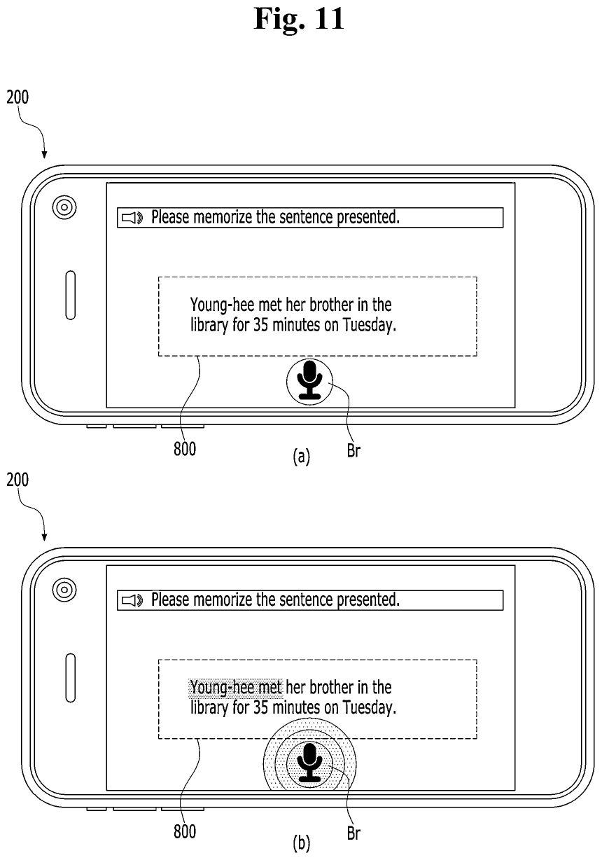

FIGS. 11 to 13 are diagrams for describing another embodiment of a method of performing a mixed test according to some embodiments of the present disclosure. In relation to FIGS. 11 to 13, contents that are redundant with those described in relation to FIGS. 1 and 2 are not described again, and differences between FIGS. 11 to 13 and FIGS. 1 and 2 are chiefly described below

Referring to FIG. 11(a), when a mixed test is performed, the processor 110 of the device 100 may cause a first screen included in a sentence 800 to be displayed in the user terminal 200.

For example, a plurality of sentences may be stored in the storage 120 of the device 100. In this case, the plurality of sentences may be sentences generated according to the six-fold principle (5W1H) by using different words. In addition, the lengths of the plurality of sentences may be different from each other. The processor 110 may select one of the plurality of sentences stored in the storage 120, and may control the communication unit 130 to transmit a signal for displaying the sentence to the user terminal 200. When receiving the signal through the communication unit 230, the processor 210 of the user terminal 200 may control the display unit 250 to display the sentence included in the signal.

As another example, a plurality of words may be stored in the storage 120 of the device 100. In this case, the plurality of words may be words having different word classes and different meanings. The processor 110 of the device 100 may generate a sentence consistent with the six-fold principle (5W1H) by combining at least some of a plurality of words based on a preset algorithm. The processor 110 may control the communication unit 130 to transmit, to the user terminal 200, a signal to display the generated sentence. When receiving the signal through the communication unit 230, the processor 210 of the user terminal 200 may control the display unit 250 to display a sentence included in the signal.

As another example, a plurality of sentences may be stored in the storage 220 of the user terminal 200. In this case, the plurality of sentences may be sentences generated according to the six-fold principle using different words. In addition, the lengths of the plurality of sentences may be different from each other. The processor 110 of the device 100 may transmit, to the user terminal 200, a signal to display a first screen including a sentence. In this case, the processor 210 of the user terminal 200 may control the display unit 250 to select and display any one sentence among the plurality of sentences stored in the storage 220.

As another example, a plurality of words may be stored in the storage 220 of the user terminal 200. In this case, the plurality of words may be words having different word classes and different meanings. The processor 110 of the device 100 may transmit, to the user terminal 200, a signal to display a first screen including a sentence. In this case, the processor 210 of the user terminal 200 may generate a sentence consistent with the six-fold principle (5W1H) by combining at least some of the plurality words stored in the storage 220 based on a preset algorithm. In addition, the processor 210 may control the display unit 250 to display the generated sentence.

The aforementioned embodiments are merely examples for description of the present disclosure, and the present disclosure is not limited thereto.

In an embodiment of the present disclosure, a first screen including the sentence 800 may include a recording button Bᵣ. In this case, the recording button Bᵣ may be displayed in the state in which a touch input to the recording button has been deactivated for a preset time.

When a preset time elapses, the processor 210 of the user terminal 200 may activate the touch input to the recording button Bᵣ.

For example, the processor 110 of the device 100 may confirm whether a preset time has elapsed from a time point at which the first screen is displayed. When recognizing that the preset time has elapsed from the time point at which the first screen including the sentence 800 was displayed, the processor 110 may transmit, to the user terminal 200, a signal to activate the recording button Bᵣ. When receiving the signal, the user terminal 200 may activate the touch input to the recording button Bᵣ.

As another example, the processor 210 of the user terminal 200 may confirm whether a preset time has elapsed from a time point at the first screen including the sentence 800 was displayed. When recognizing that the preset time has elapsed from the time point at which the first screen including the sentence 800 was displayed, the processor 210 may activate the touch input to the recording button Bᵣ.

However, the above examples are intended to describe some embodiments of the present disclosure, and the present disclosure is not limited thereto.

According to some embodiments of the present disclosure, the color of at least one word that constitutes the sentence included in the first screen may be sequentially changed regardless of the activation of a touch input to the recording button Bᵣ.

For example, when a preset time (e.g., 1 to 2 seconds) has elapsed after the first screen was displayed in the user terminal 200, the color of at least one word that constitutes the sentence included in the first screen may be changed in order. In this case, the touch input to the recording button Bᵣ may be activated or deactivated.

More specifically, the processor 110 may check whether a preset time has elapsed after the first screen was displayed in the user terminal 200. In addition, when recognizing that the preset time has elapsed, the processor 110 may control the communication unit 130 to transmit, to the user terminal 200, a signal to change at least one color that constitutes the sentence included in the first screen. In this case, when receiving the signal, the processor 210 of the user terminal 200 may control the display unit 250 to sequentially change the color of at least one word that constitutes the sentence included in the first screen. However, a method of sequentially changing the color of at least one word that constitutes the sentence included in the first screen is not limited to the above example.

As another example, the processor 110 may cause the color of at least one word that constitutes the sentence included in the first screen to be sequentially changed immediately after the first screen was displayed in the user terminal 200. In this case, the signal to display the first screen may include a signal to sequentially change at least one color that constitutes the sentences included in the first screen, and, when the user terminal 200 displays the first screen, the color of at least one word that constitutes the sentences included in the first screen may be sequentially changed. In this case, a touch input to the recording button Bᵣ may be activated or deactivated.

As still another example, the touch input to the recording button Bᵣ included in the first screen may maintain an activated state from the beginning. After the first screen was displayed in the user terminal 200, when recognizing that a touch input to the recording button Bᵣ is detected, the processor 110 may cause the color of one word to change in sequence so that the color of at least one word that constitutes the sentence included in the first screen may be sequentially changed.

More specifically, when detecting a touch input to the recording button Bᵣ included in the first screen, the processor 210 of the user terminal 200 may control the communication unit 230 to transmit information, indicating that a touch on the recording button Bᵣ has been performed, to the device 100. When receiving the information from the user terminal 200 through the communication unit 130, the processor 110 of the device 100 may recognize that a touch input to the recording button Bᵣ has been detected. In addition, the processor 110 may control the communication unit 130 to transmit, to the user terminal 200, a signal to change at least one color that constitutes the sentence included in the first screen. When receiving the signal, the processor 210 of the user terminal 200 may control the display unit 250 to sequentially change the color of at least one word that constitutes the sentence included in the first screen. However, a method of sequentially changing the color of at least one word that constitutes the sentence included in the first screen is not limited to the above example.

According to some embodiments of the present disclosure, the first screen including the sentence 800 may include a message including contents that provide notification of a task that needs to be performed by a user through a screen that is now displayed. For example, the message may include contents indicating that the sentence 800 included in the screen needs to be memorized, but the present disclosure is not limited thereto.

According to some embodiments of the present disclosure, a sound related to a message (e.g., a voice that explains contents included in the message) may be output through the sound output unit 260 in interworking with the message being displayed. In this manner, when outputting a sound together with the message to let the user know what the user needs to do, it is possible to clearly understand what the user is currently doing. Therefore, the possibility of performing a wrong operation by a simple mistake may be reduced.

Referring to FIG. 11(b), when a touch input to the recording button Bᵣ is detected after the recording button Bᵣ is activated, the processor 210 of the user terminal 200 may control the display unit 250 so that the color of at least one word that constitutes the sentence 800 included in the first screen is sequentially changed. In this case, when the color of at least one word is sequentially changed, only the color of a text may be changed, or the color may be changed in a form in which the text is highlighted with color in as illustrated in FIG. 11(b).

For example, the processor 210 of the user terminal 200 may control the communication unit 230 to transmit, to the device 100, a specific signal according to a touch input to the recording button Bᵣ by generating the specific signal. When receiving the specific signal through the communication unit 130, the processor 110 of the device 100 may transmit, to the user terminal 200, a signal to sequentially change the color of at least one word that constitutes the sentence 800 included in the first screen. When receiving the signal through the communication unit 230, the processor 210 of the user terminal 200 may control the display unit 250 to sequentially change the color of at least one word that constitutes the sentence 800 included in the first screen.

As another example, the processor 210 of the user terminal 200 may control the communication unit 230 to transmit a signal, indicating that the recording button Bᵣ has been selected, to the device 100 in response to a touch input to the recording button Bᵣ. Next, the processor 210 of the user terminal 200 may control the display unit 250 to sequentially change the color of at least one word that constitutes the sentence 800 included in the first screen. That is, the user terminal 200 may control the display unit 250 so that the color of at least one word that constitutes the sentence 800 included in the first screen is sequentially changed immediately, without receiving a separate signal from the device 100.

The color of at least one word that constitutes the sentence 800 included in the first screen may be sequentially changed from the first word of the at least one word.

For example, if the sentence 800 included in the first screen is “Young-hee met her brother in the library for 35 minutes on Tuesday”, the processor 210 may control the display unit 250 to first change the color of the first word (“Young-hee”) of the sentence 800. In addition, the processor 210 may control the display unit 250 to change the second word so that the second word has the same color as the first word after a preset time (e.g., 1 to 2 seconds) elapses. In this manner, the processor 210 may sequentially change the colors of all of at least one word that constitutes the sentence 800 included in the first screen.

The processor 210 of the present disclosure may control the display unit 250 to sequentially change the color of at least one word of the sentence 800, when receiving a specific signal by itself or from the device 100.

When the sentence 800 is simply displayed on the first screen, a user may not read the entire sentence. However, if the color of at least one word that constitutes the sentence 800 is sequentially changed as the user touches the recording button Bᵣ as described above, the user is more likely to read the entire sentence. That is, a problem in that the second test is not properly performed because a user does not read the entire sentence 800 can be solved through the aforementioned embodiment.

According to some embodiments of the present disclosure, when a touch input to the recording button Bᵣ is detected, the recording button Bᵣ may be displayed with a preset effect added thereto. For example, an effect having a form in which a preset color spreads around the recording button Bᵣ may be added to the recording button Bᵣ.

However, the preset effect is not limited to the above example, and various effects may be added to the recording button Bᵣ. If a touch input to the recording button Bᵣ is detected as described, a user can recognize that recording is now in progress as a preset effect is added to the recording button Bᵣ.

According to some embodiments of the present disclosure, when detecting a touch input to the recording button Bᵣ, the processor 110 of the device 100 may obtain a preliminary recording file. Furthermore, the processor 110 may recognize whether voice analysis is possible through the preliminary recording file obtained from the user terminal 200. When determining that the voice analysis is impossible, the processor 110 may control at least one of the display unit 250 and the sound output unit 260 to output a preset alarm. In this case, the preset alarm may be notification related to contents indicating that recording should be performed at a quiet place.

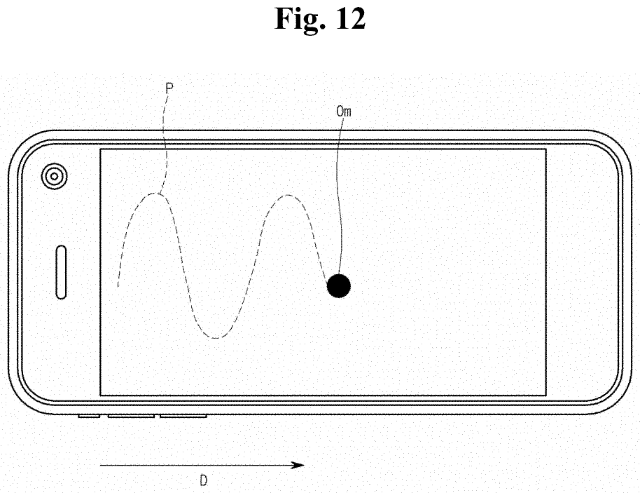

Referring to FIG. 12, the processor 110 may cause an image including an eye of a user to be obtained, in interworking with the user terminal 200 displaying a moving object Oₘ instead of the first screen including the sentence 800. In this case, the processor 110 may obtain first information related to a change in the user's gaze by analyzing the image including the eye of the user.

The moving object Oₘ displayed in the user terminal 200 may move in a specific direction D along a preset path P at a preset speed.

In an embodiment of the present disclosure, the moving object Oₘ may be an object having a specific shape of a preset size. For example, the moving object Oₘ may be a circular object having a diameter of 0.2 cm. When the object Oₘ having the shape of the aforementioned size moves, a user's gaze may move smoothly along the object.

In an embodiment of the present disclosure, the preset path P may be a path that moves with a cosine waveform or a sine waveform. Amplitude of the cosine waveform or the sine waveform may be constant, but the present disclosure is not limited thereto.

If the preset speed is 20 deg/sec to 40 deg/sec, whether a user has dementia while the user's gaze is stimulated may be accurately identified. Accordingly, the preset speed may be 20 deg/sec to 40 deg/sec, but the present disclosure is not limited thereto.

The specific direction D may be a direction from the left to right of the screen or a direction from the right to left of the screen, but the present disclosure is not limited thereto.

A mixed test may be performed by a preset round. In this case, at least one of the speed of a moving object and a direction in which the moving object moves may be changed as the round is changed. Moreover, a sentence that is displayed in the mixed test may also be changed as the round is changed.

For example, the speed of a moving object when a mixed test is performed in the first round may be slower than the speed of the moving object when the mixed test is performed in a next round. Furthermore, if the moving object has moved from left to right when the mixed test is performed in the first round, the moving object may move from left to right when the mixed test is performed in a next round. Moreover, a sentence when the mixed test is performed in the first round may be a sentence having a first length. A sentence when the mixed test is performed in a next round may be a sentence having a second length longer than the first length, but the present disclosure is not limited thereto.

Although not illustrated in FIG. 12, a screen on which the moving object is displayed may include a message including contents that provide notification of a task that needs to be performed by a user through a screen that is now displayed. For example, the message may include contents indicating that a user should stare at the moving object, but the present disclosure is not limited thereto.

According to some embodiments of the present disclosure, a sound related to a message (e.g., a voice that explains contents included in the message) may be output through the sound output unit 260 in interworking with the message being displayed. If a user is made cognizant of a task that the user has to perform by outputting a sound along with a message as described above, the user can clearly understand a task that the user should now perform. Accordingly, there is a poor possibility that the user may perform an incorrect operation by simple mistakes.

According to some embodiments of the present disclosure, the processor 110 of the device 100 may obtain an image including an eye of a user in interworking with a moving object being displayed. In addition, the processor 110 may obtain first information related to a gaze change by analyzing the image. In this case, the first information may be calculated based on a coordinate value of the pupil of the user analyzed from the image including the eye of the user. In addition, the coordinate values of the pupil may be a coordinate value of a point at which the center of the pupil is located, or may be coordinate values related to an edge of the pupil, but the present disclosure is not limited thereto.

The first information of the present disclosure may include accuracy information calculated based on a movement distance of an eye of a user and a movement distance of the moving object Oₘ; latency information calculated based on the time when the moving object Oₘ starts to move and the time when the eye of the user start to move; and speed information related to a speed at which the eye of the user move. However, when the first information includes all of the accuracy information, the latency information and the speed information, the accuracy of dementia identification may be improved.

In an embodiment of the present disclosure, the accuracy information may be information on whether the user's gaze accurately gazes at the moving object Oₘ. In this case, the accuracy information may be determined using information on a movement distance of the user's gaze and information on a movement distance of the moving object Oₘ. Specifically, as a value obtained by dividing the movement distance of the user's gaze by the movement distance of the moving object Oₘ is close to 1, it may be recognized that the user's gaze is accurately gazing at the moving object Oₘ.

In an embodiment of the present disclosure, the latency information may be information for checking a reaction speed of a user. That is, the latency information may include information on the time from a time point at which the moving object Oₘ starts to move to a time point at which an eye of the user starts to move.

In an embodiment of the present disclosure, speed information may mean a moving speed of an eye of a user. That is, the speed information may be calculated based on information on a movement distance of a user's pupils and information on the time taken for a user's pupils to move, but the present disclosure is not limited thereto. The processor 110 may calculate the speed information in various ways. For example, the processor 110 may calculate the speed information by generating a position trajectory of a user's gaze and reducing a velocity value based on a different position trajectory.

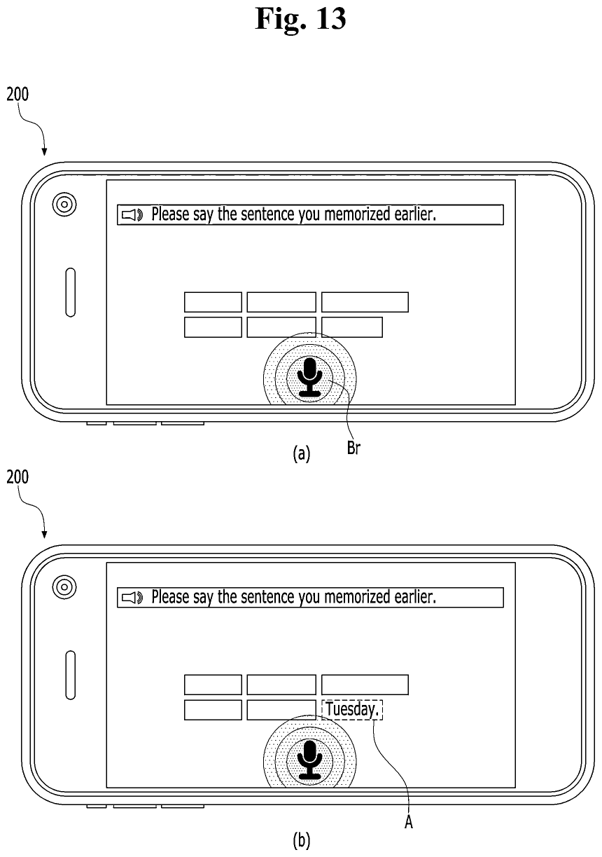

Referring to FIG. 13(a), the processor 110 may cause a recording file to be obtained, in interworking with the user terminal displaying a second screen in which a sentence is hidden. In this case, the second screen may be a screen in which at least one word segment constituting a sentence has been hidden so that how many word segments constitute the sentence can be known. If at least one word segment is divided and hidden as described above, a user may check the number of word segments. Accordingly, the user may naturally figure out a sentence that was memorized before by checking the number of word segments.

In an embodiment of the present disclosure, the second screen may include the recording button Bᵣ. However, unlike in the case in which the first screen in FIG. 11 is displayed, a touch input to the recording button Bᵣ may continue to be activated.

In some embodiments of the present disclosure, when detecting a user's touch input to the recording button Bᵣ, the processor 110 of the device 100 may control the user terminal 200 to obtain a recording file.

Specifically, when detecting a touch input to the recording button Bᵣ, the processor 210 of the user terminal 200 may obtain a recording file, including a user's voice, through the sound acquisition unit 270. The processor 210 may control the communication unit 230 to transmit the recording file to the device 100. In this case, the processor 110 of the device 100 may obtain the recording file by receiving the recording file through the communication unit 130.

When a touch input to the recording button Bᵣ is detected, the recording button Bᵣ may be displayed with a preset effect added to the recording button. For example, an effect having a form in which a preset color is spread around the recording button Bᵣ may be added to the recording button Bᵣ, but the preset effect is not limited thereto. Various effects may be added to the recording button Bᵣ. If a touch input to the recording button Bᵣ is detected and a preset effect is added to the recording button Bras described above, a user can recognize that recording is currently in progress.

According to some embodiments of the present disclosure, the second screen may include a message that informs a user of a task to be performed through a screen that is now displayed. For example, the message may include contents “Please say the sentence you memorized earlier”, but the present disclosure is not limited thereto.

According to some embodiments of the present disclosure, a sound related to a message (e.g., a voice that explains contents included in the message) may be output through the sound output unit 260 in interworking with the message being displayed. If a user is made cognizant of a task that the user has to perform by outputting a sound along with a message as described above, the user can clearly understand what task the user should perform. Accordingly, there is a poor possibility that the user may perform an incorrect operation by simple mistakes.

Referring to FIG. 13(b), the second screen may be displayed in a form in which a specific word A, among at least one word constituting a sentence, is displayed and other words except the specific word A are hidden. In this case, the specific word A may be a word including a predicate or a word disposed at the end of a sentence, but the present disclosure is not limited thereto.

As described above, when the specific word A is not hidden and is displayed on the second screen, the specific word A may be a hint for memorizing the entire sentence memorized by the user.

If a user has dementia, the user cannot memorize the entire sentence even if the specific word A is displayed. However, if a user does not have dementia, the user may memorize the entire sentence when the specific word A is displayed. Therefore, when the specific word A is displayed without being hidden by the second screen, and then the obtained recording file is analyzed and used as a digital biomarker for analyzing dementia, the accuracy of dementia identification can be increased.

According to some embodiments of the present disclosure, the processor 110 may obtain second information related to a voice of a user by using a recording file. In this case, the second information may include at least one of similarity information indicative of similarity between text data that has been converted through a voice recognition technology and original data, and voice analysis information of the user that has been analyzed based on the recording file.

A method of obtaining similarity information is first described as follows.

The processor 110 may convert a recording file into text data through a voice recognition technology. Furthermore, the processor 110 may generate similarity information indicative of similarity between the text data and original data. In this case, the original data may be the sentence 800 that is included in the first screen in FIG. 11.

Specifically, an algorithm related to a voice recognition technology (e.g., speech to text (STT)) for converting a recording file into text data may be stored in the storage 120 of the device 100. For example, the algorithm related to the voice recognition technology may be a hidden Markov model (HMM). The processor 110 may convert a recording file into text data by using the algorithm that is related to the voice recognition technology and that is stored in the storage 120. In addition, the processor 110 may generate similarity information indicative of similarity between the text data and the original text data.

A method of generating similarity information is not limited to the above example. The processor 210 of the user terminal 200 may generate similarity information in the same manner. In this case, the device 100 may obtain the similarity information by receiving the similarity information from the user terminal 200.

In an embodiment of the present disclosure, the similarity information may include information on the number of operations that are performed when text data is converted into original text data through at least one of an insertion operation, a deletion operation, and a replacement operation. In this case, as the number of operations increases, the original text data and the text data may be determined to be dissimilar to each other.

The insertion operation may refer to an operation of inserting at least one character into text data. For example, when the text data includes two characters, and the original text data includes the same characters as the text data, but includes one more character, the insertion operation may be an operation of inserting the one character included only in the original text data into the text data.

The deletion operation may mean an operation of deleting at least one character included in the text data.

For example, when the original text data includes two characters, and the text data includes the same characters as the original data, but includes one more character, the deletion operation may be an operation of deleting the one character not included in the original text data from the text data.

The replacement operation may refer to an operation of replacing at least one character included in the text data with another character. For example, when the original data includes two characters and the text data also includes two characters, but only one character in the text data is the same as that in the original data, the replacement operation may be an operation of correcting the character, included in the text data, different from the original text data to be the same as that in the original text data.

In an embodiment of the present disclosure, the voice analysis information may include at least one of user's speech speed information; and response speed information calculated based on a first time point at which the second screen is displayed and a second time point at which recording of the recording file starts, but the present disclosure is not limited thereto.

In an embodiment of the present disclosure, the speech speed information may be calculated based on information on the number of words spoken by a user and information on a total time required until the user completes the speech, but the present disclosure is not limited thereto. The processor 110 may obtain speech speed information based on various algorithms.

In an embodiment of the present disclosure, the response speed information may indicate a time taken from the first time point at which the second screen is displayed to the second time point at which recording of the recording file starts. That is, the response speed may be recognized as high when the time taken from the first time point to the second time point is short, and the response speed may be recognized as slow when the time taken from the first time point to the second time point is long.

In an embodiment of the present disclosure, result data may include at least one of accuracy information that is calculated based on a distance in which an eye of a user has moved and a distance in which a moving object has moved, latency information that is calculated based on a time point at which a moving object starts to move and a time point at which an eye of a user starts to move, speed information related to the speed at which an eye of a user has moved, similarity information indicative of similarity between text data that has been converted from a recording file through a voice recognition technology and original data, information on the speed at which a user speaks, and response speed information that is calculated based on a first time point at which a second screen is displayed and a second time point at which the recording of a recording file has started. However, in order to improve the accuracy of dementia identification, all of the accuracy information, the latency information, the speed information, the similarity information, the speaking speed information, and the response speed information may be included in the result data.

At least one element that is included in a screen related to a mixed test may include the sentence 800 and the moving object Oₘ. In this case, the size of each of the sentence 800 and the moving object Oₘ may be a preset size (e.g., a size which may be easily recognized by the aged) or more. If at least one element has a preset size or more as described above, the aged can easily perform a test.

According to some embodiments of the present disclosure, prior to the execution of a mixed test, the processor 110 may perform a preliminary test so that a user can check the contents of the mixed test. In this case, the preliminary test is performed in the same manner as the mixed test, and a detailed description thereof is omitted.

Result data that is obtained in a preliminary test may not be used to identify whether a user has dementia through the dementia identification model, but the present disclosure is not limited thereto. In order to increase the accuracy of dementia identification of the dementia identification model, result data that is obtained in a preliminary test may also be input to the dementia identification model.

FIG. 14 is a diagram for describing an embodiment of a method of displaying dementia identification result information through a preset application according to some embodiments of the present disclosure. In relation to FIG. 14, contents that are redundant with those described in relation to FIGS. 1 and 2 are not described again, and differences between FIG. 14 and FIGS. 1 to 13 are chiefly described below.

According to some embodiments of the present disclosure, when determining that a user has dementia, the processor 110 may cause dementia identification result information to be output through a preset application within the user terminal of a user. In this case, the preset application may be a messenger application, but the present disclosure is not limited thereto. The dementia identification result information may be output by the user terminal 200 through various applications.

Referring to FIG. 14, the dementia identification result information may include current state information 801 of a user and state change information 802 of the user. In this case, the current state information 801 of the user and state change information 802 of the user may be generated based on history data of the user that was obtained by performing a plurality of tests and current data of the user that is now obtained by performing a plurality of tests.

The current state information 801 of the user may be information indicating whether the cognitive ability of the user has changed compared to the past state of the user.

The state change information 802 of the user may include information on past cognitive power of the user and information on current cognitive power of the user. Furthermore, the state change information of the user may indicate a change in the cognitive power of the user in the form of a graph so that the user may intuitively recognize his or her state change, but the present disclosure is not limited thereto.

As in an embodiment of the present disclosure, if the current state information 801 of a user and the state change information 802 of the user are output through a preset application, the user can easily recognize a change in his or her cognitive ability. Accordingly, the user can prevent dementia by previously checking that his or her cognitive ability is reduced.

120 people in a normal cognitive group and 9 people in a cognitively impaired group conducted experiments in order to identify whether they had dementia by using their user terminals. The goal of this experiment was to confirm the accuracy of the pre-learned dementia identification model. Specifically, the device 100 determined whether the person had dementia based on a score value that was generated by inputting, to the dementia identification model according to an embodiment of the present disclosure, a plurality of result data obtained by performing a plurality of tests. It was confirmed that classification accuracy calculated through the aforementioned experiments was 80% or more.

According to at least one of the aforementioned several embodiments of the present disclosure, dementia may be accurately diagnosed in a way that a patient rarely feels rejection.

In an embodiment of the present disclosure, the configurations and methods of the aforementioned several embodiments of the device 100 are not limitedly applied, and all or parts of each of the embodiments may be selectively combined to allow various modifications.

Various embodiments described in the present disclosure may be implemented in a computer or similar device-readable recording medium using, for example, software, hardware, or a combination thereof.

According to hardware implementation, some embodiments described herein may be implemented using at least one of application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, and other electrical units for performing functions. In some cases, some embodiments described in the present disclosure may be implemented with at least one processor.

According to software implementation, some embodiments such as the procedures and functions described in the present disclosure may be implemented as separate software modules. Each of the software modules may perform one or more functions, tasks, and operations described in the present disclosure. A software code may be implemented as a software application written in a suitable programming language. In this case, the software code may be stored in the storage 120 and executed by at least one processor 110. That is, at least one program command may be stored in the storage 120, and the at least one program command may be executed by the at least one processor 110.

The method of identifying dementia by the at least one processor 110 of the device 100 using the dementia identification model according to some embodiments of the present disclosure may be implemented as code readable by the at least one processor in a recording medium readable by the at least one processor 110 provided in the device 100. The at least one processor-readable recording medium includes all types of recording devices in which data readable by the at least one processor 110 is stored. Examples of the at least one processor-readable recording medium includes read only memory (ROM), random access memory (RAM), CD-ROM, a magnetic tape, a floppy disk, and an optical data storage device.

Although the present disclosure has been described with reference to the accompanying drawings, this is only an embodiment and the present disclosure is not limited to a specific embodiment. Various contents that can be modified by those of ordinary skill in the art to which the present disclosure belongs also belong to the scope of rights according to the claims. In addition, such modifications should not be understood separately from the technical spirit of the present disclosure.

What is claimed is:

1. A method of identifying, by at least one processor of a device, dementia, the method comprising:
obtaining geometric features of an eye of a user by analyzing an image that comprises the eye of the user and that is obtained while a preset object is sequentially displayed in each of a plurality of regions on a screen of a user terminal for a preset time;
obtaining a plurality of result data of the user obtained by performing a plurality of tests through the user terminal;
calculating a score value by inputting the plurality of result data to a dementia identification model; and
determining whether the user has dementia based on whether the score value is greater than a first threshold value,
wherein the geometric features of the eye comprise at least one of a location of a center of a pupil of the user, a size of the pupil of the user, and a location of the eye of the user for increasing accuracy of at least one of the plurality of result data, and
the dementia identification model is not stored in storage of the user terminal and is stored in storage of the device.

2. The method of claim 1, wherein the plurality of tests comprises at least one of a Stroop test, a calculation ability test, a memory test, a gaze test, and a mixed test.

3. The method of claim 2, wherein the plurality of tests is performed in a way to display at least one element along with an output of sound data and message data that explain a method of performing each of the plurality of tests.

4. The method of claim 1, wherein the determining of whether the user has dementia based on whether the score value is greater than the first threshold value comprises:
determining that the user has dementia when the score value is greater than the first threshold value;
determining that the user has mild cognitive impairment (MCI) when the score value is greater than a second threshold value and is smaller than or equal to the first threshold value; or
determining that the user is normal when the score value is smaller than or equal to the second threshold value.

5. The method of claim 4, wherein the determining that the user has MCI comprises causing an application for improving cognitive power of the user to be executed in or downloaded to the user terminal.

6. The method of claim 4, wherein the determining of whether the user has dementia further comprises causing dementia identification result information to be output through a preset application of the user terminal of the user.

7. The method of claim 6, wherein the result information comprises current state information and state change information of the user that are generated based on history data of the user that was obtained by performing the plurality of tests and current data of the user that is now obtained by performing the plurality of tests.

8. The method of claim 1, further comprising causing hospital information generated based on information on an address of the user to be output when the score value is greater than the first threshold value.

9. The method of claim 1, further comprising obtaining information on an age and sex of the user from the user terminal before obtaining the plurality of result data,
wherein the calculating of the score value by inputting the plurality of result data to the dementia identification model comprises calculating the score value by inputting the information on the age and sex to the dementia identification model along with the plurality of result data.

10. The method of claim 1, wherein:
the dementia identification model comprises a plurality of sub-models for receiving the plurality of result data, respectively, and
the score value is an average value of a plurality of sub-score values output by the plurality of sub-models, respectively.

11. The method of claim 1, wherein:
the dementia identification model comprises a plurality of sub-models for receiving the plurality of result data, respectively, and
the calculating of the score value by inputting the plurality of result data to the dementia identification model comprises:
adding a weight of each of the plurality of sub-models to each of a plurality of sub-score values output by the plurality of sub-models; and
determining, as the score value, an average value of the plurality of sub-score values to which the weights have been added.

12. The method of claim 1, further comprising:
transmitting the score value to an external server in order to calculate dementia-related insurance premium of the user when the score value is calculated, or
transmitting dementia identification result information of the user to the external server in order to calculate dementia-related insurance premium of the user if whether the user has dementia has been determined based on the score value.

13. A computer program in which a non-transitory computer-readable storage medium has been stored, wherein the computer program performs identifying dementia when the computer program is executed in at least one processor of a device, the identifying of the dementia comprises:
obtaining geometric features of an eye of a user by analyzing an image that comprises the eye of the user and that is obtained while a preset object is sequentially displayed in each of a plurality of regions on a screen of a user terminal for a preset time;
obtaining a plurality of result data of the user obtained by performing a plurality of tests through the user terminal;
calculating a score value by inputting the plurality of result data to a dementia identification model; and
determining whether the user has dementia based on whether the score value is greater than a first threshold value,
wherein the geometric features of the eye comprise at least one of a location of a center of a pupil of the user, a size of the pupil of the user, and a location of the eye of the user for increasing accuracy of at least one of the plurality of result data, and
the dementia identification model is not stored in storage of the user terminal and is stored in storage of the device.

14. A device for identifying dementia comprising:
storage in which at least one program instruction has been stored; and
at least one processor configured to perform the at least one program instruction,
wherein the at least one processor is configured to:
obtain geometric features of an eye of a user by analyzing an image that comprises the eye of the user and that is obtained while a preset object is sequentially displayed in each of a plurality of regions on a screen of a user terminal for a preset time,
obtain a plurality of result data of the user obtained by performing a plurality of tests through the user terminal,
calculate a score value by inputting the plurality of result data to a dementia identification model, and
determine whether the user has dementia based on whether the score value is greater than a first threshold value,
wherein the geometric features of the eye comprise at least one of a location of a center of a pupil of the user, a size of the pupil of the user, and a location of the eye of the user for increasing accuracy of at least one of the plurality of result data, and
the dementia identification model is not stored in storage of the user terminal and is stored in storage of the device.
